(19) United States
(12) Patent Application Publication
(10) Pub. No.: US 2015/0216806 A1
(43) Pub. Date: Aug. 6, 2015

(54) LAXATIVE COMPOSITIONS AND METHODS FOR TREATING CONSTIPATION AND RELATED GASTROINTESTINAL DISEASES AND CONDITIONS

(71) Applicant: SALIX PHARMACEUTICALS, INC., Raleigh, NC (US)

(72) Inventor: Thomas Julius Borody, Five Dock (AU)

(21) Appl. No.: 14/424,515

(22) PCT Filed: Aug. 29, 2013
(86) PCT No.: PCT/AU2013/000973

§ 371 Date: Feb. 27, 2015

Related U.S. Application Data
(60) Provisional application No. 61/723,027, filed on Nov. 6, 2012.
(60) Provisional application No. 61/694,672, filed on Aug. 29, 2012.

(51) Int. Cl. A61K 9/48; A61K 31/437; A61K 9/50; A61K 31/655; A61K 31/454; A61K 31/538; A61K 38/08

(52) U.S. Cl. CPC A61K 9/48; A61K 31/538; A61K 31/437; A61K 38/08; A61K 31/655; A61K 31/454; A61K 9/50

(57) ABSTRACT

In alternative embodiments, the invention provides compositions, e.g., formulations or preparations, used for treating, ameliorating or preventing constipation and other disorders with related gastrointestinal symptoms. In alternative embodiments, the invention provides compositions, e.g., formulations or preparations, used for treating, ameliorating or preventing conditions which benefit from increasing or speeding bowel transit, including for example: cyclic vomiting, reflux oesophagitis, autism enteropathy, flatulence, halitosis, Chronic Fatigue Syndrome (CFS), bloating, proctalgia fugax, small intestinal bacterial overgrowth (SIBO) and large intestinal bacterial overgrowth (LIBO), chronic nausea, functional dyspepsia and bloating. In alternative embodiments, the invention provides compositions, e.g., formulations or preparations, used for treating, ameliorating or preventing a constipation, a functional constipation, Irritable Bowel Syndrome (IBS)-constipation, a diverticulosis-associated constipation, a pseudo obstruction, a slow-transit constipation, a stasis with overflow and/or a diabetic gastro-paresis. In alternative embodiments, the invention provides pharmaceuticals and products (articles) of manufacture for delivering these compositions and formulations to an individual, e.g., a human or an animal.

### TECHNICAL FIELD

This invention generally relates to medicine, pharmacology and biochemistry. In alternative embodiments, the invention provides compositions, e.g., formulations or preparations, used for treating, ameliorating or preventing constipation and other disorders with related gastrointestinal symptoms. In alternative embodiments, the invention provides compositions, e.g., formulations or preparations, used for treating, ameliorating or preventing conditions which benefit from increasing or speeding bowel transit, including for example: cyclic vomiting, reflux oesophagitis, autism enteropathy, flatulence, halitosis, Chronic Fatigue Syndrome (CFS), bloating, proctalgia fugax, small intestinal bacterial overgrowth (SIBO) and large intestinal bacterial overgrowth (LIBO), chronic nausea, functional dyspepsia and bloating. In alternative embodiments, the invention provides compositions, e.g., formulations or preparations, used for treating, ameliorating or preventing a constipation, a functional constipation, Irritable Bowel Syndrome (IBS)-constipation, a diverticulosis-associated constipation, a pseudo obstruction, a slow-transit constipation, a stasis with overflow and/or a diabetic gastro-paresis. In alternative embodiments, the invention provides pharmaceuticals and products (articles) of manufacture for delivering these compositions and formulations to an individual, e.g., a human or an animal.

### BACKGROUND

The human gastrointestinal (GI) microbiota, the “GI microbiome”, is complex and is composed of around 3.3 million nonredundant microbial genes. Most of these are bacterial species, and the entire cohort harbors between 1,000 and 1,150 prevalent bacterial species. The gastrointestinal microbiome is considered now to be a “virtual organ,” where only a small percentage of the entire cohort can be cultured and studied with respect to the metabolic pathways and activities of the bacteria. Genomic studies are easier but they do not give us the answer as to the functional capacity of various bacteria.

Being a “virtual organ,” this bacterial cohort is susceptible, like any other organ of the body, to suffer from various “organ disorders”. The most common one is infection, and so the intestinal microbiome can become infected e.g., with parasites, bacteria or viruses. Clinically, such infection can either be acute or chronic, and some examples of such acute infections are Salmonella or Shigella, and of chronic ones are Clostridium difficile, Giardia lamblia, Blastocystis hominis etc. Probably the most common infections of the gut microbiome are yet to be described and constitute what we have come to know as ‘Irritable Bowel Syndrome’ or IBS. It is now known that symptomatically infection of the gut flora does not always end up in diarrhea, but rather may be present in many forms that may be asymptomatic, or can cause diarrhea, cramping, abdominal pain, gas; and, in particular, an infection of the gut flora can also cause constipation.

For centuries constipation has been viewed as a benign condition somehow related to our diet. In recent decades the role of fiber has taken center stage and in the medical and lay public's mindset constipation is caused by ‘inadequate dietary fiber, too little exercise and lack of water intake’. Few have addressed the super-infection of the intestinal microbiome as contributory.

Constipation is a very common condition especially in the developed countries. Notwithstanding the causality and the many secondary rather than primary causes such as hypothyroidism, hypercalcaemia and various medications, e.g., narcotic derivatives, there is a clinical need for effective laxatives that do not have any long term adverse effects. In the past therapies for constipation have included the increasing of fiber intake, many and varied laxatives such as Senna, Coloxyl, exotic teas and osmotic laxatives such as sorbitol, mannitol, lactulose and polyethylene glycol and others. Various other laxatives have been used including bisacodyl and castor oil, linactolide, prucalopride and colchicine. Methylnaltrexone has also been used to antagonise opiate induced constipation. Prokinetic agents including cisapride, metoclopramide, mosapride and domperidone have also been used to increase motility in some patients. Antibiotics such as erythromycin and vancomycin have also been used.

However, in spite of giving the large numbers of various anti-constipation agents there is an indication that there is always a need for a better treatment, especially in the more severe cases of constipation. It is the object of this invention to bring to the market a more user-friendly and far more effective therapy for constipation addressing also the microbiome.

### SUMMARY

The invention provides compositions and methods for treating, ameliorating or preventing constipation, including chronic or acute constipation, and other disorders with related gastrointestinal symptoms.

In alternative embodiments, the invention provides compositions, pharmaceutical compositions or formulations, formulated for delayed or gradual enteric release, comprising at least one active agent formulated with a delayed release composition or formulation, coating, microencapsulation or encapsulation, wherein the composition comprises:
(a) (i) at least one active agent comprising a bisoxatin (or 2,2-bis(4-hydroxyphenyl)-2H-benzo[b][1,4]oxazin-3(4H)-one), or a bisoxatin acetate, or an equivalent,
wherein optionally the bisoxatin is a LAXONALIN™, a MARATAN™, a TALSIS™, or a TASIS™, and
(ii) the delayed or gradual enteric release composition or formulation, coating, microencapsulation or encapsulation;
(b) the composition, a pharmaceutical composition or a formulation of (a), formulated as a delayed or gradual enteric release composition or formulation,
wherein optionally the formulation comprises a gastro-resistant coating designed to dissolve at a pH of 7 in the terminal ileum, e.g., an active ingredient is coated with an acrylic based resin or equivalent, e.g., a methacrylic acid copolymer B, NF, such as EUDRAGIT S™, which dissolves at pH 7 or greater, e.g., comprises a multimatrix (MMX) formulation; or
(c) the composition, a pharmaceutical composition or a formulation of (a) or (b), formulated as a laxative.

In alternative embodiments, the invention provides compositions, pharmaceutical compositions or formulations, comprising:
(a) a bisoxatin (or 2,2-bis(4-hydroxyphenyl)-2H-benzo[b][1,4]oxazin-3(4H)-one), or a bisoxatin acetate, or an equivalent,
wherein optionally the bisoxatin is a LAXONALIN™, a MARATAN™, a TALSIS™, or a TASIS™, and
(b)(i) an antibiotic or an antimicrobial, wherein optionally the antibiotic or an antimicrobial is not absorbed from the lumen;
   wherein optionally the antimicrobial or antibiotic is or comprises one or more of a: glycopeptide antibiotic, wherein optionally the glycopeptide antibiotic is a vancomycin, a teicoplanin (e.g., TARGOCID™), a telavancin (e.g., VIBATIV™), a bleomycin (e.g., BLENOXANE™), a ramoplanin or a decaplanin; or, a fidaxomycin, a gentamycin, a neomycin, a streptomycin, a paromomycin, a kanamycin, a rifaximin (e.g., the extended intestinal release (EIR) rifaximin) or another rifamycin (including e.g., the rifamycin derivatives rifampicin (or rifampin), rifabutin, rifapentine and rifalazil), or an ansamycin, a geldanamycin, an ansamitocin, or an anti-protozoal agent such as nitazoxanide (e.g., DAXON™, DEXIDEX™, KIDONAX™, MITAFAR™, PACOVANTON™, PARAMIX™), a furazolidone (e.g., FUROXONE™, DEPENDAL-M™), a nitroimidazole or metronidazole (e.g., a 5-nitroimidazole, FLAGYL™), a nifuroxazide (e.g., AMBATROL™, ANTINAL™, BACIFURANE™, DIAFURYL™) or a bismuth (e.g., bismuth subsalicylate), also including various groups of antibiotics such as a penicillin (e.g., penicillin G, procaine penicillin, benzathine penicillin or penicillin V), a macrolide (e.g., erythromycin, a clarithromycin, a dirithromycin (e.g., DYNABAC™), a roxithromycin (e.g., XTHROCIN™, ROXL-150™, ROXO™, SURLID™), a telithromycin (e.g., KETEK™) or an azithromycin such a ZITHROMAX™, AZITHROCIN™), a tetracycline, a cephalosporin, a carbapenem (e.g., imipenem, a meropenem such as MONAN™, MERONEM™), a monobactam, a lincosamide or a clindamycin (e.g., DALACIN™), a quinolone (e.g., a fluoroquinolone) and/or a sulphonamide, a fradicin (e.g., NEOBIOTIC™), or an equivalent thereof or a combination thereof, or
   wherein optionally the antimicrobial or antibiotic is or comprises one or more of an aminoglycoside antibiotic (e.g., a gentamycin, a neomycin, a streptomycin, a paromomycin and/or a kanamycin), amphenicol, ansamycin, beta-lactam (β-lactam), carbapenem, cephalosporin, cephamycin, monobactam, oxacephem, a lincosamide antibiotic (e.g., clindamycin, lincomycin), a macrolide antibiotic (e.g., an azithromycin, clarithromycin, dirithromycin, erythromycin), glycopeptide antibiotic (e.g., a vancomycin, teicoplanin, telavancin, bleomycin, ramoplanin and/or a decaplanin), a polypeptide antibiotic (e.g., actinomycin, such as actinomycin D; bacitracin; bacitracin), tetracycline, or a 2,4-diaminopyrimidine class antibiotic, a clavacin (also known as clairformin, claviform, expansine, clavatin, expansin, gigantin, leucopin, patuline or patulin), or an equivalent thereof or a combination thereof;
   (ii) a colchicine or an equivalent thereof;
   (iii) an anti-inflammatory agent, wherein optionally the inflammatory agent comprises or is a 4 or a 5-amino-salicylate, an olsalazine (e.g., DIPENTUM™), a mesalazine (also known as mesalamine or a 5-aminosalicylic acid (5-ASA), e.g., ASACOL™ or LIALDA™), a sulfasalazine (e.g., AZULFIDINE™, SALAZOPYRIN™ or SULAZINE™), and/or a balsalazide (e.g. COLAZAL™ or COLAZIDE™), or an equivalent thereof or a combination thereof,
   wherein optionally any of these alternative embodiments can be administered at about 90 to 1000 mgm per day;
   (iv) a fiber product, wherein optionally the fiber comprises a psyllium or an ispaghula or an equivalent thereof;
   (v) a prokinetic agent, wherein optionally the prokinetic agent comprises a cisapride (e.g., PREPULSID™, PROPULSID™), a mosapride, a prucalopride (e.g., RESOLOR™, RESOTRAN™), a metoclopramide and/or a domperidone (e.g., MOTILIUM™, MOTILLIUM™, MOTINORM COSTI™, NOMIT™) or an equivalent thereof or a combination thereof;
   (vi) a sulphate, wherein optionally the sulphate comprises a sodium sulphate, a picosulphate, a sodium picosulphate or equivalent, a potassium sulphate or a magnesium sulphate or an equivalent thereof or a combination thereof;
   (vii) a phosphate, wherein optionally the phosphate comprises a sodium phosphate or an equivalent thereof;
   (viii) a laxative, wherein optionally the laxative comprises a bisacodyl (e.g., a DULCOLAX™, a DUROLAX™, a FLEET™, an ALOPHEN™, or a CORRECTOL™), a docusate sodium, a poloxamer, a sennoside, a lactulose, a sorbitol, a sugar, a sterculia or frangula, a paraffin oil or an equivalent thereof or a combination thereof;
   (ix) at least one osmotic laxative, wherein optionally the osmotic laxative comprises a sorbitol, mannitol, lactulose and/or a polyethylene glycol, wherein optionally the polyethylene glycol (PEG) is a PEG 3350, or MIRALAX™;
   (x) at least one non-osmotic purgative, wherein optionally the non-osmotic purgative comprises one or more of a colchicine, a mineral oil, an aloe, a bisacodyl, a sodium picosulfate, a casanthranol, a cascara, a castor oil, a danthron, a dehydrocholic acid, a phenolphthalein, a sennoside, a docusate, a bethanachol, a misoprostol, cisapride, norcisapride, paraffin, rhein, and/or tegaserod; and/or further comprising at least one bulk-forming purgative, which optionally comprises a methylcellulose, sodium carboxymethyl cellulose, bran, psyllium, sterculia, and/or testa ispaghula;
   (xi) at least one: anti-narcotic agent and/or a neural stimulant, wherein optionally the anti-narcotic agent comprises a naloxone hydrochloride (e.g., NARCAN™, NALONE™, NARCANTI™) (e.g., administered at e.g., about 20 to 50 mgm per unit dosage), a naltrexone (e.g., REVIA™, DEPADE™, VIVITROL™), a methylnaltrexone bromide, a nalmefene glucuronide, or an equivalent), and optionally the neural stimulant comprises a neostigmine, a physostigmine, a pyridostigmine or a pyridostigmine bromide;
   (xii) at least one opiate inhibitor or opiate antagonist, wherein optionally the opiate inhibitor or opiate antagonist is a methylnaltrexone bromide, a naltrexone (e.g., REVIA™, DEPADE™, VIVITROL™), or a nalmefene glucuronide;
   (xiii) at least one acid suppressant, antacid and/or proton pump inhibitor, wherein optionally the acid suppressant is an H2 Receptor Antagonist, wherein optionally the H2 Receptor Antagonist is a cimetidine (e.g., TAGAMET™), a ranitidine (e.g., ZANTAC™), or an equivalent, wherein optionally the Proton Pump Inhibitor is an omeprazole (e.g., LOSEC™, ANTRA™, GASTROLOC™, MOPRAL™, OMEPRAL™, PRILOSEC™), an esameprazole (e.g., NEXIUM™), a pantoprazole (e.g., SOMAC™, TECTA™, PANTOLOC™, PROTIUM™ PROTONIX™) and equivalents; or
   (xiv) one or more probiotics, wherein optionally the probiotic comprises a cultured or stool-extracted microorganism or bacteria, or a bacterial component, and optionally the bacteria or bacterial component comprises or is derived from a Bacteroidetes, a Firmicutes, a Lactobacilli, a Bifidobacteria, an E coli, a Strep fecalis and equivalents; or
   (xv) a Biofilm Disrupting Compound, wherein optionally the biofilm disrupting compound comprises an enzyme, a deoxyribonuclease (DNase), N-acetylcysteine, an alginate lyase, glycoside hydrolase dispersin B; Quorum-sensing inhibitors e.g., ribonucleic acid III inhibiting peptide, Salvadora persica extracts, Competence-stimulating peptide, Patulin and penicillic acid; peptides—cathelicidin-derived peptides, small lytic peptide, PTP-7, Nitric oxide, neo-emulsions; ozone, lytic bacteriophages, lactoferrin, xylitol hydrogel, synthetic iron chelators, cranberry components, curcumin, silver nanoparticles, Acetyl-11-keto-β-boswellic acid (AKBA), barley coffee components, probiotics, sinefungin, S-adenosylmethionine, S-adenosyl-homocysteine, Delisea furanones, N-sulfonyl homoserine lactones or any combination thereof; or
   (xvi) any two of (i) to (xv), any three of (i) to (xv), or any four or more of (i) to (xv), or any combination thereof,
wherein optionally the composition, a pharmaceutical composition or formulation is formulated as a delayed or gradual enteric release composition or formulation, and optionally the formulation comprises a gastro-resistant coating designed to dissolve at a pH of 7 in the terminal ileum, e.g., an active ingredient is coated with an acrylic based resin or equivalent, e.g., a poly(meth)acrylate, e.g. a methacrylic acid copolymer B, NF, such as EUDRAGIT S™ (Evonik Industries AG, Essen, Germany), which dissolves at pH 7 or greater, e.g., comprises a multimatrix (MMX) formulation,
wherein optionally the composition, a pharmaceutical composition or formulation is formulated as a laxative.

In alternative embodiments, the compositions, pharmaceutical compositions or formulations of the invention can further comprise at least one vitamin, mineral and/or dietary supplement, wherein optionally the vitamin comprises a thiamine, riboflavin, nicotinic acid, pantothenic acid, pyridoxine, biotin, folic acid, vitamin B₁₂, lipoic acid, ascorbic acid, vitamin A, vitamin D, vitamin E, vitamin K, a choline, a carnitine, and/or an alpha, beta and/or gamma carotene.

In alternative embodiments the bisoxatin, bisoxatin acetate or equivalent comprises:
between about 0.10 to about 1000 milligrams (mg), or between about 0.10, 0.20, 0.30, 0.40, 0.50, 0.60, 0.70, 0.80. 0.90, 1.0, 2, 3, 4, 5, 6, 7, 8, 9, 10, 15, 20, 21, 22, 23, 24, 25, 30, 31, 32, 33, 34, 35, 36, 37, 38, 39, 40, 40, 45, 50, 55 to 60 milligram (mg) to about 100, 150, 200, 250, 300, 350, 400, 450, 500, 550, 600, 650, 700, 750, 800, 850, 900, 950 or 1000 or more milligrams (mg), or
between about 5 milligrams (mg) to about 15 milligrams (mg), or the composition comprises about 0.10, 0.20, 0.30, 0.40, 0.50, 0.60, 0.70, 0.80. 0.90, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 54, 70, 75, 80, 85, 90, 95, 100, 105, 110, 115, 120, 130, 140, 150, 160, 170, 180, 190, 200, 210, 220, 230, 240, 250, 275, 300, 350, 400, 450 or 500 or more mgs of:
the bisoxatin (or 2,2-bis(4-hydroxyphenyl)-2H-benzo[b][1,4]oxazin-3(4H)-one), or the bisoxatin acetate, or equivalent.

In alternative embodiments, the bisoxatin, bisoxatin acetate or equivalent comprises:
between about 0.10, 0.20, 0.30, 0.40, 0.50, 0.60, 0.70, 0.80. 0.90, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45 to 50 milligram (mg) to about 100, 150, 200, 250, 300, 350, 400, 450, 500, 550, 600, 650, 700, 750, 800, 850, 900, 950 or 1000 or more milligrams (mg), or
between about 5 milligrams (mg) to about 15 milligrams (mg), or the composition comprises about 0.10, 0.20, 0.30, 0.40, 0.50, 0.60, 0.70, 0.80. 0.90, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 54, 70, 75, 80, 85, 90, 95, 100, 105, 110, 115, 120, 130, 140, 150, 160, 170, 180, 190, 200, 210, 220, 230, 240, 250, 275, 300, 350, 400, 450 or 500 or more mgs,
of a bisoxatin (or 2,2-bis(4-hydroxyphenyl)-2H-benzo[b][1,4]oxazin-3(4H)-one), or bisoxatin acetate, or equivalent.

In alternative embodiments, the bisoxatin, bisoxatin acetate or equivalent comprises:
between about 0.10, 0.20, 0.30, 0.40, 0.50, 0.60, 0.70, 0.80. 0.90, 1.0, 2, 3, 4, 5, 6, 7, 8, 9 or 10 mg to about 0.10, 0.20, 0.30, 0.40, 0.50, 0.60, 0.70, 0.80. 0.90, 1.0, 2, 3, 4, 5, 6, 7, 8, 9 or 10 or more grams (g) bisoxatin (or 2,2-bis(4-hydroxyphenyl)-2H-benzo[b][1,4]oxazin-3(4H)-one), or bisoxatin acetate, or equivalent; or
between about 75, 80, 85, 90 or 100 mg to about 150 to 200 mg bisoxatin (or 2,2-bis(4-hydroxyphenyl)-2H-benzo[b][1,4]oxazin-3(4H)-one), or bisoxatin acetate, or equivalent, or
between about 100, 110, 120, 130, 140 or 150 mg to about 1, 2, 3, 4 or 4.5 g or more bisoxatin (or 2,2-bis(4-hydroxyphenyl)-2H-benzo[b][1,4]oxazin-3(4H)-one), or bisoxatin acetate, or equivalent.

In alternative embodiments, the bisoxatin, bisoxatin acetate or equivalent comprises:
between about 10, 20, 30, 40, 50, 75, 80, 85, 90, 100 or 150 mg to about 100, 150, 200, 250, 300, 350, 400, 450, or 500 or more mg, or between about 50, 75, 80, 85, 90, 100 or 150 mg to about 150 to 200 mg, or
between about 100 to 250 mg, or between about 100, 110, 120, 130, 140 or 150 mg to about 1, 2, 3, 4 or 4.5 g or more, of:
a bisoxatin (or 2,2-bis(4-hydroxyphenyl)-2H-benzo[b][1,4]oxazin-3(4H)-one), or bisoxatin acetate, or an equivalent.

In alternative embodiments, the bisoxatin, bisoxatin acetate or equivalent comprises or is a LAXONALIN™, a MARATAN™, a TALSIS™, or a TASIS™.

In alternative embodiments, the compositions, pharmaceutical compositions or formulations of the invention can further comprise at least one dispersal agent, buffering agent, sweetening agent, debittering agent, flavoring agent, pH stabilizer, acidifying agent, preservative, desweetening agent and/or coloring agent.

In alternative embodiments, the delayed or gradual enteric release composition or formulation, coating, microencapsulation dr encapsulation comprises or is formulated as: an enteric coated tablet, multi-particulate or multilayered tablet or capsule; a gelatin, a soft gelatin or equivalent thereof; a vinyl or a polyvinyl acetate phthalate or equivalent thereof; an ACRYL-EZE™, SURETERIC™, NUTRATERIC II™®, PHTHALAVIN®. (Colorcon, Inc. Harleysville, Pa.); a hydroxypropylmethylcellulose (HPMC), a high viscosity grade HPMC, or an ultra-high viscosity grade HPMC; a polyvinylpyrrolidone (PVP) or a PVP-K90; a cellulose, a microcrystalline cellulose (MCC), a methylcellulose, a hydroxy methylcellulose, a hydroxy propyl methylcellulose (HPMC), or an ethyl cellulose; a copolymer of ethyl acrylate, a poly(meth)acrylate, e.g. a methacrylic acid copolymer B, a methyl methacrylate and/or a methacrylic acid ester with quaternary ammonium groups; EUDRAGIT® RL PO™; EUDRAGIT® RL 100™ (Evonik Industries AG, Essen, Germany).

In alternative embodiments, the delayed or gradual enteric release composition or formulation, coating, microencapsulation or encapsulation comprises: cellulose acetate phthalate, hydroxypropyl methylcellulose phthalate, polyvinyl acetate phthalate, hydroxypropyl methylcellulose acetate succinate, cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride, ethyl methyacrylate-methylmethacrylate-chlorotrimethylammonium ethyl acrylate copolymer, natural resins, zein, shellac, copal collophorium or an acrylic copolymer, or any combination or mixture thereof.

In alternative embodiments, the delayed or gradual enteric release composition or formulation, coating, microencapsulation or encapsulation comprises or further comprising a sustained-release coating, and optionally the sustained-release coating comprises a wax mixed with a glyceryl monostearate, a stearic acid, a palmitic acid, a glyceryl monopalmitate, a cetyl alcohol, a shellac, a zein, an ethylcellulose, an acrylic resin, a cellulose acetate or a silicone elastomer or any combination or mixture thereof.

In alternative embodiments, the compositions, pharmaceutical compositions or formulations of the invention can further comprise a water-soluble salt, and optionally the salt comprises a salt consisting of a calcium salt, a calcium carbonate, a calcium acetate, a citrate salt, a calcium citrate, a magnesium salt, a magnesium sulphate, a magnesium citrate, a monobasic sodium phosphate, dibasic sodium phosphate, and/or tribasic sodium phosphate, a magnesium phosphate, a sodium salt, a sodium sulphate, a sodium chloride, a sodium gluconate, a sodium citrate, a sodium aspartate, a potassium salt, a potassium gluconate, a potassium tartrate, a potassium chloride, an acetate salt, an adipate salt, an alginate salt, an aspartate salt, a benzoate salt, a benzenesulfonate salt, a bisulfate salt, a butyrate salt, a camphorate salt, a camphor sulfonate salt, a digluconate salt, a glycerophosphate salt, a hemisulfate salt, a heptanoate salt, a hexanoate salt, a fumarate salt, a hydrochloride salt, a hydrobromide salt, a hydroiodide salt, a 2-hydroxyethansulfonate (isothionate) salt, a lactate salt, a maleate salt, a methane sulfonate salt, a nicotinate salt, a 2-naphthalene sulfonate salt, an oxalate salt, a palmitoate salt, a pectinate salt, a persulfate salt, a 3-phenylpropionate salt, a picrate salt, a pivalate salt, a propionate salt, a succinate salt, a tartrate salt, a thiocyanate salt, a phosphate salt, a glutamate salt, a bicarbonate salt, a p-toluenesulfonate salt, a undecanoate salt, or any equivalent salt, or any salt as described in “Handbook of Pharmaceutical Salts: Properties, Selection and Use”, Weinheim, N.Y.: VHCA; Wiley-VCH, 2002, or any mixture thereof.

In alternative embodiments, a composition, pharmaceutical composition or formulation of the invention is manufactured, labeled or formulated as a preparation, a pharmaceutical or a formulation for human or animal use, wherein optionally the animal use is for a veterinary use.

In alternative embodiments, a composition, pharmaceutical composition or formulation of the invention is manufactured, labeled or formulated as a powder, a lyophilized or freeze-dried product, a liquid, a suspension, a spray, a gel, a hydrogel, a geltab, a semisolid, a tablet, a lozenge, a sachet or a capsule; or is manufactured, labeled or formulated as a food, a drink, a yogurt, a candy, a lollypop (lolly) or a paste.

In alternative embodiments, a composition, pharmaceutical composition or formulation of the invention further comprises a defoaming agent, a surfactant agent, a lubricant, an acid neutralizer, a marker, a cell marker and/or a contrast agent, and optionally the surfactant agent comprises a simethicone or any mixture of polydimethylsiloxane and silica gel, or equivalent, and optionally the lubricant comprises a magnesium stearate, a hyaluronic acid, a glycerol and/or a silicone, and/or the lubricant comprises an encapsulating material, wherein the encapsulating material acts as a capsule or covering for a preparation of the composition; or, wherein the deforming agent comprises a silicone and/or a glycerol, and optionally the acid neutralizer comprises a water-soluble acid neutralizer, which optionally comprises a tromethamine, a meglumine, a sodium bicarbonate, a sodium carbonate, or any combination thereof, or the acid neutralizer comprises a water-insoluble acid neutralizer, which optionally comprises a magnesium hydroxide, an aluminum hydroxide, a dihydroxy aluminum sodium carbonate, a calcium carbonate, and any combination thereof.

In alternative embodiments, a composition, pharmaceutical composition or formulation of the invention further comprises an antibiotic or an antimicrobial, wherein optionally the antibiotic or an antimicrobial is not absorbed from the lumen, e.g., a nonabsorbable antibiotic that provides activity locally in the gut due to its negligible systemic absorption such as a rifamycin or a rifaximin. The antimicrobial or antibiotic can be, or comprises, one or more of a: glycopeptide antibiotic, wherein optionally the glycopeptide antibiotic is a vancomycin, a teicoplanin (e.g., TARGOCID™), a telavancin (e.g., VIBATIV™), a bleomycin (e.g., BLENOXANE™), a ramoplanin or a decaplanin; or, a fidaxomycin, a gentamycin, a neomycin, a streptomycin, a paromomycin, a kanamycin, a rifaximin (e.g., the extended intestinal release (EIR) rifaximin, e.g., a XIFAXAN™) or a rifamycin (including e.g., the rifamycin derivatives rifampicin (or rifampin), rifabutin, rifapentine and rifalazil), or an ansamycin, a geldanamycin, an ansamitocin, or an anti-protozoal agent such as nitazoxanide (e.g., DAXON™, DEXIDEX™, KIDONAX™, MITAFAR™, PACOVANTON™, PARAMIX™), a furazolidone (e.g., FUROXONE™, DEPENDAL-M™), a nitroimidazole or metronidazole (e.g., a 5-nitroimidazole, FLAGYL™), a nifuroxazide (e.g., AMBATROL™, ANTINAL™, BACIFURANE™, DIAFURYL™) or a bismuth (e.g., bismuth subsalicylate), also including various groups of antibiotics such as a penicillin (e.g., penicillin G, procaine penicillin, benzathine penicillin or penicillin V), a macrolide (e.g., erythromycin, a clarithromycin, a dirithromycin (e.g., DYNABAC™), a roxithromycin (e.g., XTHROCIN™, ROXL-150™, ROXO™, SURLID™), a telithromycin (e.g., KETEK™) or an azithromycin such a ZITHROMAX™, AZITHROCIN™), a tetracycline, a cephalosporin, a carbapenem (e.g., imipenem, a meropenem such as MONAN™, MERONEM™), a monobactam, a lincosamide or a clindamycin (e.g., DALACIN™), a quinolone (e.g., a fluoroquinolone), a sulphonamide, and/or a fradicin (e.g., NEOBIOTIC™), or an equivalent thereof or a combination thereof. The antimicrobial or antibiotic can be or comprises one or more of an aminoglycoside antibiotic (e.g., a gentamycin, a neomycin, a streptomycin, a paromomycin and/or a kanamycin), amphenicol, ansamycin, beta-lactam (β-lactam), carbapenem, cephalosporin, cephamycin, monobactam, oxacephem, a lincosamide antibiotic (e.g., clindamycin, lincomycin), a macrolide antibiotic (e.g., an azithromycin, clarithromycin, dirithromycin, erythromycin), glycopeptide antibiotic (e.g., a vancomycin, teicoplanin, telavancin, bleomycin, ramoplanin and/or a decaplanin), a polypeptide antibiotic (e.g., actinomycin, such as actinomycin D; bacitracin; bacitracin), tetracycline, or a 2,4-diaminopyrimidine class antibiotic, a clavacin (also known as clairformin, claviform, expansine, clavatin, expansin, gigantin, leucopin, patuline or patulin), or an equivalent thereof or a combination thereof.

In alternative embodiments, a composition, pharmaceutical composition or formulation of the invention further comprises a colchicine or an equivalent thereof.

In alternative embodiments, a composition, pharmaceutical composition or formulation of the invention further comprises an anti-inflammatory agent, wherein optionally the inflammatory agent comprises or is a 4 or a 5-amino-salicylate, an olsalazine (e.g., DIPENTUM™), a mesalazine (also known as mesalamine (e.g., ASACOL™ or LIALDA™) or a 5-aminosalicylic acid (5-ASA)), a sulfasalazine (e.g., AZULFIDINE™, SALAZOPYRIN™ or SULAZINE™), and/or a balsalazide (e.g. COLAZAL™ or COLAZIDE™), or an equivalent thereof or a combination thereof,
wherein optionally any of these alternative embodiments can be administered at about 90 to 1000 mgm per day.

In alternative embodiments, a composition, pharmaceutical composition or formulation of the invention further comprises a fiber product, wherein optionally the fiber comprises a psyllium or an ispaghula or an equivalent thereof.

In alternative embodiments, a composition, pharmaceutical composition or formulation of the invention further comprises a prokinetic agent, wherein optionally the prokinetic agent comprises a cisapride (e.g., PREPULSID™, PROPULSID™), a mosapride, a prucalopride (e.g., RESOLOR™, RESOTRAN™), a metoclopramide and/or a domperidone (e.g., MOTILIUM™, MOTILLIUM™, MOTINORM COSTI™, NOMIT™) or an equivalent thereof or a combination thereof.

In alternative embodiments, a composition, pharmaceutical composition or formulation of the invention further comprises a sulphate, wherein optionally the sulphate comprises a sodium sulphate, a picosulphate, a sodium picosulphate or equivalent, a potassium sulphate or a magnesium sulphate or an equivalent thereof or a combination thereof.

In alternative embodiments, a composition, pharmaceutical composition or formulation of the invention further comprises a phosphate, wherein optionally the phosphate comprises a sodium phosphate or an equivalent thereof.

In alternative embodiments, a composition, pharmaceutical composition or formulation of the invention further comprises a laxative, wherein optionally the laxative comprises a bisacodyl (e.g., a DULCOLAX™, a DUROLAX™, a FLEET™, an ALOPHEN™, or a CORRECTOL™), a docusate sodium, a poloxamer, a sennoside, a lactulose, a sorbitol, a sugar, a sterculia or frangula, a paraffin oil or an equivalent thereof or a combination thereof.

In alternative embodiments, a composition, pharmaceutical composition or formulation of the invention further comprises at least one non-osmotic purgative, wherein optionally the non-osmotic purgative comprises one or more of a colchicine, a mineral oil, an aloe, a bisacodyl, a sodium picosulfate, a casanthranol, a cascara, a castor oil, a danthron, a dehydrocholic acid, a phenolphthalein, a sennoside, a docusate, a bethanachol, a misoprostol, cisapride, norcisapride, paraffin, rhein, and/or tegaserod; and/or further comprising at least one bulk-forming purgative, which optionally comprises a methylcellulose, sodium carboxymethyl cellulose, bran, psyllium, sterculia, and/or testa ispaghula.

In alternative embodiments, a composition, pharmaceutical composition or formulation of the invention further comprises at least one: anti-narcotic agent and/or a neural stimulant, wherein optionally the anti-narcotic agent comprises a naloxone hydrochloride (e.g., NARCAN™, NALONE™, NARCANTI™) (e.g., administered at e.g., about 20 to 50 mgm per unit dosage), a naltrexone (e.g., REVIA™, DEPADE™, VIVITROL™), a methylnaltrexone bromide, a nalmefene glucuronide, or an equivalent), and optionally the neural stimulant comprises a neostigmine, a physostigmine, a pyridostigmine or a pyridostigmine bromide.

In alternative embodiments, a composition, pharmaceutical composition or formulation of the invention further comprises at least one acid suppressant, antacid and/or proton pump inhibitor, wherein optionally the acid suppressant is an H2 Receptor Antagonist, wherein optionally the H2 Receptor Antagonist is a cimetidine (e.g., TAGAMET™), a ranitidine (e.g., ZANTAC™), or an equivalent, wherein optionally the Proton Pump Inhibitor is an omeprazole (e.g., LOSEC™, ANTRA™, GASTROLOC™, MOPRAL™, OMEPRAL™, PRILOSEC™), an esameprazole (e.g., NEXIUM™), a pantoprazole (e.g., SOMAC™, TECTA™, PANTOLOC™, PROTIUM™ PROTONIX™) and equivalents.

In alternative embodiments, a composition, pharmaceutical composition or formulation of the invention further comprises one or more probiotics, wherein optionally the probiotic comprises a cultured or stool-extracted microorganism or bacteria, or a bacterial component, and optionally the bacteria or bacterial component comprises or is derived from a Bacteroidetes, a Firmicutes, a Lactobacilli, a Bifidobacteria, an E coli, a Strep fecalis and equivalents.

In alternative embodiments, a composition, pharmaceutical composition or formulation of the invention further comprises at least one osmotic laxative, wherein optionally the osmotic laxative comprises a sorbitol, mannitol, lactulose and/or a polyethylene glycol, wherein optionally the polyethylene glycol (PEG) is a PEG 3350, or MIRALAX™.

In alternative embodiments, a composition, pharmaceutical composition or formulation of the invention further comprises at least one opiate inhibitor or opiate antagonist, wherein optionally the opiate inhibitor or opiate antagonist is a methylnaltrexone bromide, a naltrexone (e.g., REVIA™, DEPADE™, VIVITROL™), or a nalmefene glucuronide.

In alternative embodiments, a composition, pharmaceutical composition or formulation of the invention further comprises a Biofilm Disrupting Compound, wherein optionally the biofilm disrupting compound comprises an enzyme, a deoxyribonuclease (DNase), N-acetylcysteine, an alginate lyase, glycoside hydrolase dispersin B; Quorum-sensing inhibitors e.g., ribonucleic acid III inhibiting peptide, Salvadora persica extracts, Competence-stimulating peptide, Patulin and penicillic acid; peptides—cathelicidin-derived peptides, small lytic peptide, PTP-7, Nitric oxide, neo-emulsions; ozone, lytic bacteriophages, lactoferrin, xylitol hydrogel, synthetic iron chelators, cranberry components, curcumin, silver nanoparticles, Acetyl-11-keto-β-boswellic acid (AKBA), barley coffee components, probiotics, sinefungin, S-adenosylmethionine, S-adenosyl-homocysteine, Delisea furanones, N-sulfonyl homoserine lactones or any combination thereof.

In alternative embodiments, a composition, pharmaceutical composition or formulation of the invention further comprises at least one dispersal agent, buffering agent, sweetening agent, debittering agent, flavoring agent, pH stabilizer, acidifying agent, preservative, desweetening agent and/or coloring agent.

In alternative embodiments, a composition, pharmaceutical composition or formulation of the invention further comprises at least one vitamin, mineral and/or dietary supplement, wherein optionally the vitamin comprises a thiamine, riboflavin, nicotinic acid, pantothenic acid, pyridoxine, biotin, folic acid, vitamin B₁₂, lipoic acid, ascorbic acid, vitamin A, vitamin D, vitamin E, vitamin K, a choline, a carnitine, and/or an alpha, beta and/or gamma carotene.

The invention provides compositions, pharmaceutical compositions or formulations comprising:
a bisoxatin, a bisoxatin acetate or equivalent, and an antibiotic or at least two antibiotics, wherein optionally one or both or all of the antibiotics is a nonabsorbable antibiotic, e.g., a streptomycin, neomycin, a gentamycin, a rifaximin (e.g., a XIFAXAN™) and/or a vancomycin;
a bisoxatin, a bisoxatin acetate or equivalent, and an antibiotic (e.g., a nonabsorbable antibiotic, e.g., a streptomycin, neomycin, a gentamycin, a rifaximin, e.g., a XIFAXAN™) and colchicine;
a bisoxatin, a bisoxatin acetate or equivalent, and an antibiotic (e.g., a nonabsorbable antibiotic, e.g., a streptomycin, neomycin, a gentamycin, a rifaximin) and an acid inhibitor, wherein optionally the bisoxatin, antibiotic and acid inhibitor combination comprises a bisoxatin, a rifaximin and an omeprazole;
a bisoxatin, a bisoxatin acetate or equivalent, and a probiotic and a balsalazide;
a bisoxatin and a rifaximin and a balsalazide;
wherein optionally the pharmaceutical composition or formulation is formulated for delayed or gradual enteric release.

The invention provides compositions, pharmaceutical compositions or formulations formulated for a pediatric indication comprising:
a bisoxatin, a bisoxatin acetate or equivalent, and an anti-inflammatory agent;
a bisoxatin, a bisoxatin acetate or equivalent, and an olsalazine (e.g., DIPENTUM™); or
a bisoxatin, a bisoxatin acetate or equivalent, and a balsalazide (e.g. COLAZAL™ or COLAZIDE™), a 4 and 5-amino-salicylate, a mesalazine (e.g., LIALDA™) or a sulfasalazine (e.g., AZULFIDINE™, SALAZOPYRIN™ or SULAZINE™),
wherein optionally the composition is formulated as a chewable lolly (lollypop), candy, ice, ice cream or yoghurt,
and optionally the pharmaceutical composition or formulation is formulated for delayed or gradual enteric release.

The invention provides compositions, pharmaceutical compositions or formulations formulated for a narcotic use (use with or after use of a narcotic) indication comprising:
a bisoxatin, a bisoxatin acetate or equivalent, and an opiate inhibitor or an opiate antagonist;
a bisoxatin, a bisoxatin acetate or equivalent, and a methylnaltrexone bromide;
a bisoxatin, a bisoxatin acetate or equivalent, and a naltrexone (e.g., REVIA™, DEPADE™, VIVITROL™); or
a bisoxatin, a bisoxatin acetate or equivalent, and a nalmefene glucuronide.

The invention provides compositions, pharmaceutical compositions or formulations formulated for a Parkinson's disease indication comprising:
a bisoxatin, a bisoxatin acetate or equivalent; a laxative; and, an antibiotic;
a bisoxatin, a bisoxatin acetate or equivalent; a colchicine; and, an antibiotic; or,
a bisoxatin, a bisoxatin acetate or equivalent; a colchicine; and, a vancomycin,
and optionally the pharmaceutical composition or formulation is formulated for delayed or gradual enteric release.

The invention provides compositions, pharmaceutical compositions or formulations formulated for an acute constipation comprising:
a bisoxatin, a bisoxatin acetate or equivalent, and an osmotic laxative;
a bisoxatin acetate or equivalent; and, a sorbitol, mannitol, lactulose and/or polyethylene glycol, wherein optionally the polyethylene glycol (PEG) is a PEG 3350, or MIRALAX™;
wherein optionally the composition is formulated as a sachet,
and optionally the pharmaceutical composition or formulation is formulated for delayed or gradual enteric release.

The invention provides compositions, pharmaceutical compositions or formulations formulated for a pain, or for Non-Specific Abdominal Pain Syndrome, comprising;
a bisoxatin, a bisoxatin acetate or equivalent; an osmotic laxative; and, an antibiotic; or,
a bisoxatin, a bisoxatin acetate or equivalent; a sorbitol, mannitol, lactulose and/or polyethylene glycol; and, a rifaximine, wherein optionally the polyethylene glycol (PEG) is a PEG 3350, or MIRALAX™,
wherein optionally the composition is formulated as a sachet,
and optionally the pharmaceutical composition or formulation is formulated for delayed or gradual enteric release.

The invention provides compositions, pharmaceutical compositions or formulations formulated for Inflammatory Bowel Disease with constipation, comprising:
a bisoxatin, a bisoxatin acetate or equivalent, and an anti-inflammatory agent; or
a bisoxatin, a bisoxatin acetate or equivalent; and, a balsalazide (e.g. COLAZAL™ or COLAZIDE™), a 4 and 5-amino-salicylate, an olsalazine (e.g., DIPENTUM™), a mesalazine (e.g., LIALDA™) or a sulfasalazine (e.g., AZULFIDINE™, SALAZOPYRIN™ or SULAZINE™),
and optionally the pharmaceutical composition or formulation is formulated for delayed or gradual enteric release.

The invention provides articles or products of manufacture, blister packages, lidded blisters or blister cards or packets, clamshells, trays or shrink wraps, or kits, comprising one or combination of compositions, pharmaceutical compositions or formulations of the invention.

The invention provides pharmaceutical compositions, preparations, formulations, foods, candies, yogurts, ices, ice creams, lozenges, feeds, supplements, food supplements, additives or food additives, comprising a composition of the invention, or an article or product of manufacture or kit of the invention, wherein optionally the pharmaceutical composition, preparation or formulation is manufactured, labeled or formulated as a liquid, a suspension, a gel, a geltab, a semisolid, a tablet, a sachet, a lozenge or a capsule, or as an enteral formulation. The pharmaceutical composition or a formulation can be manufactured with an enteric coating, or an encapsulating or a multilayered material.

In alternative embodiments, the pharmaceutical compositions, preparations or formulations, articles or products of manufacture, blister packages, lidded blisters or blister cards or packets, clamshells, trays or shrink wraps, or kits of the invention, or the pharmaceutical composition, preparation or formulation of the invention, are manufactured, labeled or formulated for the amelioration or treatment of:
a constipation, functional constipation, Irritable Bowel Syndrome (IBS)-constipation, diverticulosis-associated constipation, pseudo obstruction, slow-transit constipation, stasis with overflow and diabetic gastro-paresis, or
any condition which can benefit from speeding bowel transit, including cyclic vomiting, reflux oesophagitis, autism enteropathy, flatulence, halitosis, Chronic Fatigue Syndrome (CFS), bloating, proctalgia fugax, small intestinal bacterial overgrowth (SIBO) and large intestinal bacterial overgrowth (LIBO), chronic nausea, functional dyspepsia, and bloating.

The invention provides methods for the amelioration, treatment and/or prevention of:
a constipation, a chronic constipation, an acute constipation, functional constipation, Irritable Bowel Syndrome (IBS)-constipation, diverticulosis-associated constipation, pseudo obstruction, slow-transit constipation, stasis with overflow and diabetic gastro-paresis, or
any condition which can benefit from speeding bowel transit, including cyclic vomiting, reflux oesophagitis, autism enteropathy, flatulence, halitosis, Chronic Fatigue Syndrome (CFS), bloating, proctalgia fugax, small intestinal bacterial overgrowth (SIBO) and large intestinal bacterial overgrowth (LIBO), chronic nausea, functional dyspepsia, and bloating,
to an individual in need thereof, comprising: administering composition of the invention, the article or product of manufacture, a blister package, a lidded blister or a blister card or packet, a clamshell, a tray or a shrink wrap, or a kit of the invention, or the pharmaceutical composition, preparation or formulation of the invention,
wherein optionally the bisoxatin (or 2,2-bis(4-hydroxyphenyl)-2H-benzo[b][1,4]oxazin-3(4H)-one), bisoxatin acetate, or equivalent is administered at a dosage of between about 1 to 360 mgm a day, or is administered at a dosage of 1.0, 2, 3, 4, 5, 6, 7, 8, 9, 10, 15, 20, 21, 22, 23, 24, 25, 30, 31, 32, 33, 34, 35, 36, 37, 38, 39, 40, 40, 45, 50, 55, 60, 70, 80, 90, 100, 125, 150, 175, 200, 225, 250, 275, 300, 325, 350 or 360 milligram (mg) a day,
wherein optionally the unit dosage of the bisoxatin (or 2,2-bis(4-hydroxyphenyl)-2H-benzo[b][1,4]oxazin-3(4H)-one), bisoxatin acetate, or equivalent is between about 20 to 120 mgm per unit dosage, or between about 20 to 125 mgm per unit dosage, or the unit dosage is about 20, 21, 22, 23, 24, 25, 30, 31, 32, 33, 34, 35, 36, 37, 38, 39, 40, 40, 45, 50, 55, 60, 70, 75, 80, 90, 100, 110, 115, 120 or 125 mgm per unit dosage,
and optionally the capsules, tablets, sachets, geltabs, lozenges or other unit dosage formulations can be administered in a dosage (e.g., a unit dosage) regimen of from between 1 and 6 per day long term for the duration of the treatment, e.g., for the duration of the constipation.

In alternative embodiments, the constipation or bloating is due to at least one of: travel; change in daily routine; lack of exercise; immobility caused by injury, illness, or aging; dehydration; irritable bowel syndrome; pregnancy; diabetes; hypothyroidism; hypercalcemia; cancer of the colon or rectum; uterine prolapse; vaginal vault prolapse; rectal prolapse; scarring from surgery; injury of the colon or rectum; Parkinson's disease; multiple sclerosis; stroke; hemorrhoids or anal fissures; delaying bowel movements; anxiety; depression; eating disorders; and/or obsessive-compulsive disorder, coeliac disease, muscular dystrophy, myotonic dystrophy, non-specific abdominal pain, or a neurological condition or any cause of constipation.

The invention provides packages or kits comprising combination of at least two formulations, wherein one (a first) formulation contained in a first container (e.g., a bottle or blister pack or equivalent) and a second formulation is contained in a second container (e.g., a bottle or blister pack or equivalent), and the formulations are designed to be taken in sequence as part of a treatment or a regimen, wherein a patient is administered or instructed to take the contents of a first container (e.g., a bottle, blister pack, and the like) comprising a composition of the invention, an article or product of manufacture, a blister package, a lidded blister or a blister card or packet, a clamshell, a tray or a shrink wrap, or a kit of the invention, or a pharmaceutical composition, preparation or formulation of the invention, before the contents of a second container.

The invention provides a yogurt, a candy, a lollypop, a lozenge, an ice, an ice cream, a milk or a milkshake, a “frosty”, “snow-cone”, or other ice-based mix, comprising: a composition of the invention, an article or product of manufacture, a blister package, a lidded blister or a blister card or packet, a clamshell, a tray or a shrink wrap, or a kit of the invention, or a pharmaceutical composition, preparation or formulation of the invention.

The invention provides uses of a composition of the invention, an article or product of manufacture, a blister package, a lidded blister or a blister card or packet, a clamshell, a tray or a shrink wrap, or a kit of the invention, or a pharmaceutical composition, preparation or formulation of the invention, in the manufacture (preparation) of a medicament for the treatment of
a constipation, functional constipation, a chronic constipation, an acute constipation, Irritable Bowel Syndrome (IBS)-constipation, diverticulosis-associated constipation, pseudo obstruction, slow-transit constipation, stasis with overflow and diabetic gastro-paresis, or
any condition which can benefit from speeding bowel transit, including cyclic vomiting, reflux oesophagitis, autism enteropathy, flatulence, halitosis, Chronic Fatigue Syndrome (CFS), bloating, proctalgia fugax, small intestinal bacterial overgrowth (SIBO) and large intestinal bacterial overgrowth (LIBO), chronic nausea, functional dyspepsia, and bloating,
and optionally the medicament, e.g., the capsules, tablets, sachets, geltabs, lozenges or other unit dosage formulations, are manufactured for administration in a dosage (e.g., a unit dosage) regimen of from between 1 and 6 per day long term for the duration of the treatment, e.g., for the duration of the constipation.

The invention provides therapeutic combinations of drugs for ameliorating, diminishing, treating, blocking or preventing:
a constipation, a chronic constipation, an acute constipation, functional constipation, Irritable Bowel Syndrome (IBS)-constipation, diverticulosis-associated constipation, pseudo obstruction, slow-transit constipation, stasis with overflow and diabetic gastro-paresis, or
any condition which can benefit from speeding bowel transit, including cyclic vomiting, reflux oesophagitis, autism enteropathy, flatulence, halitosis, Chronic Fatigue Syndrome (CFS), bloating, proctalgia fugax, small intestinal bacterial overgrowth (SIBO) and large intestinal bacterial overgrowth (LIBO), chronic nausea, functional dyspepsia, and bloating,
comprising:
(a) a composition, pharmaceutical composition or formulation of the invention; or
(b) a composition, pharmaceutical composition or formulation of the invention, and
   (i) an antibiotic such as a penicillin, a macrolide, a tetracycline, a cephalosporin, a carbapenem, a monobactam, a glycopeptide, a lincosamide, a quinolone, a fradicin (e.g., NEOBIOTIC™), a streptothricin, a streptomycin, a neomycin, a gentamycin, a grisein, a neomycin, a candicidin, a candidin, and/or a sulphonamide;
   (ii) a colchicine, a 4 or a 5-amino-salicylate, an olsalazine, a mesalazine (e.g., LIALDA™), a azulfidine and/or a balsalazide;
   (iii) a fiber product and/or a psyllium;
   (iv) a prokinetic agent, a cisapride, a mosapride, a prucalopride, a metoclopramide and/or a domperidone;
   (v) a sulphate, a sodium sulphate, a picosulphate, a potassium sulphate and/or a magnesium sulphate;
   (vi) a phosphate and/or a sodium phosphate;
   (vii) a laxative, a bisacodyl, a docusate sodium, a poloxamer, a sennoside, a lactulose, a sorbitol, a sugar, a sterculia, a frangula and/or a paraffin oil
   (viii) an anti-narcotic agent, a naloxone, a naloxone hydrochloride, a naltrexone, a methylnaltrexone, a methylnaltrexone bromide, a nalmefene glucuronide, a nalmefene, a cyclazocine, a cyclorphan, an oxilorphan nalorphine and/or a levallorphan or a pharmaceutically acceptable salt thereof or any mixture thereof;
   (ix) a neural stimulant, a neostigmine, a physostigmine, a pyridostigmine and/or a pyridostigmine bromide; and/or
   (x) an acid suppressant, an acid reflux agent, an H2 Receptor Antagonist, a cimetidine, a ranitidine, a Proton Pump Inhibitor, an omeprazole, an esameprazole, a pantoprazole and/or an antacid.

The invention provides methods for the amelioration, treatment and/or prevention of:
a constipation, a chronic constipation, an acute constipation, a functional constipation, an Irritable Bowel Syndrome (IBS)-constipation, a diverticulosis-associated constipation, a pseudo obstruction, a slow-transit constipation, a stasis with overflow and/or a diabetic gastro-paresis, or
any condition which can benefit from speeding bowel transit, including cyclic vomiting, a reflux oesophagitis, an autism enteropathy, a flatulence, a halitosis, a Chronic Fatigue Syndrome (CFS), a bloating, a proctalgia fugax, a small intestinal bacterial overgrowth (SIBO) or a large intestinal bacterial overgrowth (LIBO), a chronic nausea, functional dyspepsia, and/or a bloating,
to an individual in need thereof, comprising: administering a therapeutic combination of drugs of the invention,
wherein optionally the bisoxatin (or 2,2-bis(4-hydroxyphenyl)-2H-benzo[b][1,4]oxazin-3(4H)-one), bisoxatin acetate, or equivalent is administered at a dosage of between about 1 to 360 mgm a day, or is administered at a dosage of 1.0, 2, 3, 4, 5, 6, 7, 8, 9, 10, 15, 20, 21, 22, 23, 24, 25, 30, 31, 32, 33, 34, 35, 36, 37, 38, 39, 40, 40, 45, 50, 55, 60, 70, 80, 90, 100, 125, 150, 175, 200, 225, 250, 275, 300, 325, 350 or 360 milligram (mg) a day,
wherein optionally the unit dosage of the bisoxatin (or 2,2-bis(4-hydroxyphenyl)-2H-benzo[b][1,4]oxazin-3(4H)-one), bisoxatin acetate, or equivalent is between about 20 to 120 mgm per unit dosage, or between about 20 to 125 mgm per unit dosage, or the unit dosage is about 20, 21, 22, 23, 24, 25, 30, 31, 32, 33, 34, 35, 36, 37, 38, 39, 40, 40, 45, 50, 55, 60, 70, 75, 80, 90, 100, 110, 115, 120 or 125 mgm per unit dosage,
and optionally the capsules, tablets, sachets, geltabs, lozenges or other unit dosage formulations can be administered in a dosage (e.g., a unit dosage) regimen of from between 1 and 6 per day long term for the duration of the treatment, e.g., for the duration of the constipation.

The details of one or more embodiments of the invention are set forth in the description below. Other features, objects, and advantages of the invention will be apparent from the description and from the claims.

All publications, patents, patent applications cited herein are hereby expressly incorporated by reference for all purposes.

### DETAILED DESCRIPTION

In alternative embodiments, the invention provides compositions formulated for delayed of gradual enteric release comprising at least one active agent formulated with a delayed release composition or formulation, coating or encapsulation, wherein the composition comprises: at least one active agent comprising a bisoxatin (or 2,2-bis(4-hydroxyphenyl)-2H-benzo[b][1,4]oxazin-3(4H)-one), or a bisoxatin acetate, or a LAXONALIN™, a MARATAN™, a TALSIS™, or a TASIS™, or an equivalent, and the delayed or gradual enteric release composition or formulation, coating or encapsulation.

In alternative embodiments the bisoxatin (or 2,2-bis(4-hydroxyphenyl)-2H-benzo[b][1,4]oxazin-3(4H)-one), bisoxatin acetate, or equivalent is administered at a dosage of between about 1 to 360 mgm a day, or is administered at a dosage of 1.0, 2, 3, 4, 5, 6, 7, 8, 9, 10, 15, 20, 21, 22, 23, 24, 25, 30, 31, 32, 33, 34, 35, 36, 37, 38, 39, 40, 40, 45, 50, 55, 60, 70, 80, 90, 100, 125, 150, 175, 200, 225, 250, 275, 300, 325, 350 or 360 milligram (mg) a day. In alternative embodiments the unit dosage of the bisoxatin (or 2,2-bis(4-hydroxyphenyl)-2H-benzo[b][1,4]oxazin-3(4H)-one), bisoxatin acetate, or equivalent is between about 20 to 120 mgm per unit dosage, or between about 20 to 125 mgm per unit dosage, or the unit dosage is about 20, 21, 22, 23, 24, 25, 30, 31, 32, 33, 34, 35, 36, 37, 38, 39, 40, 40, 45, 50, 55, 60, 70, 75, 80, 90, 100, 110, 115, 120 or 125 mgm per unit dosage.

Exemplary capsules of the invention (or other unit dosage formulations, e.g., tablets, sachets, geltabs, lozenges and the like) can be administered in a dosage (e.g., a unit dosage) regimen of from between 1 and 6 per day long term for the duration of the treatment, e.g., for the duration of the constipation.

In alternative embodiments, the invention provides compositions for treating, ameliorating and/or preventing those various gastrointestinal (GI) disorders which come under the title of constipation, functional constipation, IBS-constipation, diverticulosis-associated constipation, pseudo obstruction, slow-transit constipation, stasis with overflow and diabetic gastro-paresis, and methods for treating, ameliorating and/or preventing these diseases and conditions.

In alternative embodiments, compositions and methods of the invention also can be used for treating or ameliorating patients or individuals presenting conditions which benefit from speeding bowel transit including cyclic vomiting, reflux oesophagitis, autism enteropathy, flatulence, halitosis, Chronic Fatigue Syndrome (CFS), bloating, proctalgia fugax, small intestinal bacterial overgrowth (SIBO) and large intestinal bacterial overgrowth (LIBO), chronic nausea, functional dyspepsia, and bloating.

In alternative embodiments, compositions and methods of the invention comprise use of bisoxatin using a special delivery or by combining this molecule, bisoxatin and equivalent, co-administered with one, two or more molecules or substances as a combination to achieve and enhance the clinical result.

The novel medication would be clinically positioned for chronic daily use for both adult and children's formulations. It could also be used as a self-adjusting dosing protocol given that the severity of human constipation can vary from person to person, day to day, and even within sub-groups of various disorders. The severity of constipation can fluctuate on day to day basis, possibly due to diet, exercise, menstruation cycle, associated medications, dehydration and travel. Furthermore this self-adjusting dosing is useful in that the patient can be the immediate measure of response to the treatment. By learning from the stool frequency, quality and quantity that the treatment can give the patient can increase or decrease the dosing. Hence the patient can learn to self-manage the severity of constipation by adjusting the medication often driven by the other factors mentioned above that may influence the microbiome of that patient.

In alternative embodiments, a composition or method of the invention comprises as an active agent bisoxatin alone, e.g., formulated (or presented) (as with any exemplary composition of the invention) as an enteric-coated medication (e.g., a gel tab, a tablet, a capsule or a microparticle), or as a microencapsulated product, e.g., in a sachet with or without enteric coating. In alternative embodiments, this or any exemplary composition of the invention can also be used (or formulated) as a suppository, liquid syrup or an enema.

In alternative embodiments, a composition or method of the invention is a double therapy which combines use of bisoxatin with one or more anti-infective agents. For example, in alternative embodiments, the bisoxatin compound can be combined with a glycopeptide antibiotic (e.g., such as a vancomycin, a teicoplanin (e.g., TARGOCID™), a telavancin (e.g., VIBATIV™), a bleomycin (e.g., BLENOXANE™), a ramoplanin or a decaplanin), a fidaxomycin (e.g., DIFICID™, DIFICLIR™), a gentamycin, a neomycin, a streptomycin, a paromomycin, a kanamycin, a rifaximin (e.g., the extended intestinal release (EIR) rifaximin) and other rifamycins (including the rifamycin derivatives rifampicin (or rifampin), rifabutin, rifapentine and rifalazil.), or an ansamycin, a geldanamycin, an ansamitocin, or an anti-protozoal agent such as nitazoxanide (e.g., DAXON™, DEXIDEX™, KIDONAX™, MITAFAR™, PACOVANTON™, PARAMIX™), a furazolidone (e.g., FUROXONE™, DEPENDAL-M™), a nitroimidazole or metronidazole (e.g., a 5-nitroimidazole, FLAGYL™), a nifuroxazide (e.g., AMBATROL™, ANTINAL™, BACIFURANE™, DIAFURYL™) or a bismuth (e.g., bismuth subsalicylate), also including various groups of antibiotics such as a penicillin (e.g., penicillin G, procaine penicillin, benzathine penicillin or penicillin V), a macrolide (e.g., erythromycin, a clarithromycin, a dirithromycin (e.g., DYNABAC™), roxithromycin (e.g., XTHROCIN™, ROXL-150™, ROXO™, SURLID™), a telithromycin (e.g., KETEK™) or an azithromycin such a ZITHROMAX™, AZITHROCIN™), a tetracycline, a cephalosporin, a carbapenem (e.g., imipenem, a meropenem such as MONAN™, MERONEM™), a monobactam, a lincosamide or a clindamycin (e.g., DALACIN™), a quinolone (e.g., a fluoroquinolone) and/or a sulphonamide.

In alternative embodiments and in some conditions depending on the intended use, alternative effective combinations are: bisoxatin+rifaximin to address e.g., dysmotility and/or associated bacterial overgrowth of the microbiome; bisoxatin+vancomycin, which is very poorly absorbed and can be given safely long term.

In alternative embodiments, the glycopeptide antibiotic, e.g., vancomycin, is administered at between about 20 to 500 mgm per day, or at between about 20 to 3000 mgm per day, or optionally administered at about 125 mgm to 3 gram a day. In alternative embodiments, the vancomycin is administered at between about 500 mgm per unit dosage, optionally administered at between about 500 to 2000 mgm per day. In alternative embodiments, dosages of the antibiotics are administered at their usual clinically relevant dosages per day and unit dosages.

In alternative embodiments delivery methods include tablets, capsules, granules, enteric-coated or uncoated, graded release or mass-release. They may be suppositories, enemas, sachets, chewable ‘lolly’ preparations, chewing gum preparations, sublingual tablets or membranes, liquid preparations or may be delivered in yoghurts, chocolates or similar foods especially for children. In alternative embodiments delivery sites may be range from the oral mucosa though to the colonic mucosa and several sites may be used simultaneously e.g. small and large bowel.

In alternative embodiments having two or more active agents, the agents will possess co-activity and the combinations will exert a more efficacious clinical result than if one was to use them as mono-therapy. In alternative embodiments co-therapy can be advantageous depending on the condition treated and patient's condition. For example, efficacy of 2 or more active agents can be present in the medication but at lower concentrations to minimize adverse effects. Also, several components can act on constipation and its complications from different mechanisms. These include not only slow transit but also pain, bloat, nausea end loss of urge. Such an approach enrolls several drug effects not possessed by a single drug necessary to cover as many mechanisms and symptoms as possible. For example, in one embodiment, bisoxatin combined with an antibiotic addresses dysmotility plus bacterial overgrowth often resulting in methane production and bloat (see e.g., Pimentel et al. (2006) Am. J. Physiol. Gastrointest. Liver Physiol. 290G:1089-5). Any antibiotics can be used in alternative embodiments, but for some conditions and patients a preferred choice includes agents (e.g., medications) which are not absorbed from the lumen.

In alternative embodiments, compositions and methods of the invention comprise use of a bisoxatin combined other active agents which complement its therapeutic effects, e.g., its motility effects, for the treatment, amelioration or prevention of, e.g., constipation, cyclic vomiting, reflux oesophagitis, autism enteropathy, flatulence, halitosis, Chronic Fatigue Syndrome (CFS), bloating, proctalgia fugax, small intestinal bacterial overgrowth (SIBO) and large intestinal bacterial overgrowth (LIBO), chronic nausea, functional dyspepsia and/or bloating. In alternative embodiments these other active agents (used in combination with a bisoxatin) include, for example, colchicine, anti-inflammatory agents such as e.g., 4 and 5-amino-salicylates, such as e.g., olsalazine (e.g., DIPENTUM™), mesalazine (also known as mesalamine or 5-aminosalicylic acid (5-ASA), e.g., ASACOL™ or LIALDA™), sulfasalazine (e.g., AZULFIDINE™, SALAZOPYRIN™ or SULAZINE™), and balsalazide. All of these alternative embodiments can be administered at about 90 to 1000 mg per day.

In alternative embodiments, compositions and methods of the invention comprise use of a bisoxatin combined with one or more active agents (used in combination with a bisoxatin) including, for example: various fiber products (e.g., psyllium or ispaghula); a prokinetic agent (a gastroprokinetic agent, a gastrokinetic or a prokinetic), e.g., including cisapride (e.g., PREPULSID™, PROPULSID™), mosapride, prucalopride (e.g., RESOLOR™, RESOTRAN™), metoclopramide, and domperidone (e.g., MOTILIUM™, MOTILLIUM™, MOTINORM COSTI™, NOMIT™)); a sulphate (e.g., a sodium sulphate, a picosulphate, a potassium sulphate or a magnesium sulphate); a phosphate (e.g., a sodium phosphate).

In alternative embodiments, compositions and methods of the invention comprise use of a bisoxatin combined with a laxative, e.g., a bisacodyl (e.g., a DULCOLAX™, a DUROLAX™, a FLEET™, an ALOPHEN™, or a CORRECTOL™), a docusate sodium, a poloxamer, a sennoside, a lactulose, a sorbitol and/or a sugar, a sterculia/frangula, a paraffin oil, and the like.

In alternative embodiments, compositions and methods of the invention comprise use of a bisoxatin combined with an anti-narcotic agent (e.g., naloxone hydrochloride (e.g., NARCAN™, NALONE™, NARCANTI™), which can be administered e.g., at about 20 to 50 mgm per unit dosage), naltrexone (e.g., REVIA™, DEPADE™, VIVITROL™), methylnaltrexone bromide, nalmefene glucuronide, and the like). In alternative embodiments, compositions and methods of the invention comprise use of a bisoxatin combined with a neural stimulant, e.g., neostigmine (e.g., PROSTIGMIN™, VAGOSTIGMIN™, physostigmine, pyridostigmine, and pyridostigmine bromide.

The bisoxatin may also be combined with acid suppressants as acid reflux is common in constipation, where in alternative embodiments these agents include H2 Receptor Antagonists, e.g., cimetidine (e.g., TAGAMET™), ranitidine (e.g., ZANTAC™) and others, and/or Proton Pump Inhibitors, e.g., omeprazole (e.g., LOSEC™, ANTRA™, GASTROLOC™, MOPRAL™, OMEPRAL™, PRILOSEC™) and esameprazole (e.g., NEXIUM™), pantoprazole (e.g., SOMAC™, TECTA™, PANTOLOC™, PROTIUM™ PROTONIX™) and others, and various antacids.

In alternative embodiments, compositions and methods of the invention comprise use of a bisoxatin combined with one or more probiotics, e.g., cultured or stool-extracted. Various bacterial components can be used, including Bacteroidetes, Firmicutes, Lactobacilli, Bifidobacteria, E coli, Strep fecalis and others. These can function in inhibiting the methanogens, Clostridia and other contributory causal bacterial commensals and pathogens.

In alternative embodiments of compositions and methods of the invention, all components may be administered in amounts ranging from 0.001 mg to 500 grams.

In alternative embodiments, compositions and methods of the invention comprise use of a bisoxatin in a triple combination that can be used to deliver lower drug doses but greater spread of activity. In these embodiments, the bisoxatin is combined with any components of the previously listed groups: for example, bisoxatin+2 antibiotics, (e.g., rifaximin+vancomycin); bisoxatin+antibiotic+colchicine; bisoxatin+antibiotic+acid inhibitor (e.g., bisoxatin+rifaximin+omeprazole); or, bisoxatin+probiotic/s+balsalazide; bisoxatin+rifaximin+balsalazide. In alternative embodiments, the colchicine is administered at a unit dosage of between about 0.5 to 6 mgm per day.

In alternative embodiments, compositions and methods of the invention are formulated as combinations made to suit a particular condition, patient population, clinical result desired, and the like, for example:
Pediatric indication: bisoxatin+olsalazine as chewable lolly or available in yoghurt.
Narcotic Use indication: bisoxatin+methylnaltrexone bromide.
Parkinson's Disease indication: isoxatin+colchicine+vancomycin.
Acute Constipation, e.g., in emergency room—as a sachet of bisoxatin+sorbitol.
Non-Specific Abdominal Pain Syndrome—Bisoxatin+sorbitol+rifaximine.
Inflammatory Bowel Disease with constipation—Bisoxatin+balsalazide.

In alternative embodiments, the invention provides compositions and methods using low dosages of a bisoxatin. In alternative embodiments, the “low” dosages of bisoxatin are at or less than about 0.10, 0.20, 0.30, 0.40, 0.50, 0.60, 0.70, 0.80. 0.90, 1.0, 2, 3, 4, 5, 6, 7, 8, 9, 10, 15, 20, 21, 22, 23, 24, 25, 30, 31, 32, 33, 34, 35, 36, 37, 38, 39, 40, 40, 45, 50, 55 to 60 milligram (mg) per dosage. Sugars such as mannitol, sorbitol and/or lactulose, or equivalents, can be to enhance a laxative action.

In alternative embodiments, silicones are used to manufacture formulations or preparations of the invention; including polymers that comprising silicon together with carbon, hydrogen, oxygen or other chemical elements. In one aspect, gelatin capsules incorporating glycerol are used; they can further assist as a lubricant and defoamer.

Exemplary capsules of the invention (or other unit dosage formulations, e.g., tablets, sachets, geltabs, lozenges and the like) can be administered in a dosage (e.g., a unit dosage) regimen of from between 1 and 6 (e.g., 1, 2, 3, 4, 5 or 6) unit dosage formulations per day long term for e.g., a constipation, or, or an adjusted number or unit dosages, or total dosages, as required for an individual's (e.g., patient's) needs.

For example, in constipated or bloated patients capsule (or other unit dosage formulations) numbers can be increased—and in one embodiment, is done so during the actual preparation by the patient, so incorporating a ‘graded-dosage’ concept. In those patients with soft, frequent motions they can decrease the number. The type of fluids ingested by the patient with the capsules can be at the patient's discretion (e.g., tea, Diet Coke, water, sugar-free juices or drinks).

In one embodiment, the invention provides a dry composition to be encapsulated (or otherwise manufactured in a comparable unit dosage formulations, e.g., a geltab) for administration in a dosage (e.g., a unit dosage) regimen of from between 1 and 6 (e.g., 1, 2, 3, 4, 5 or 6) unit dosage formulations per day long term for e.g., a constipation, or, or an adjusted number or unit dosages, or total dosages, as required for an individual's (e.g., patient's) needs.

### Bisoxatin

In alternative embodiments, the invention provides compositions comprising a bisoxatin (or 2,2-bis(4-hydroxyphenyl)-2H-benzo[b][1,4]oxazin-3(4H)-one), or bisoxatin acetate, or equivalent. In alternative embodiments, a formulation or composition of the invention comprises between about 10 mg to about 1, 2, 3, 4 or 5 or more grams (g) bisacodyl, or between about 75, 80, 85, 90 or 100 mg to about 150 to 200 mg (e.g., for a normal patient) bisacodyl, or between about 100, 110, 120, 130, 140 or 150 mg to about 1, 2, 3, 4 or 4.5 g or more bisacodyl for a constipated patient.

In alternative embodiments, the bisoxatin is LAXONALIN™, MARATAN™, TALSIS™, TASIS™.

### Additional Optional Ingredients

### Bisacodyl

In alternative embodiments, the invention provides compositions further comprising a bisacodyl, or pyridin-2-ylmethanediyl)dibenzene-4,1-diyl diacetate, or 4,4′-(pyridin-2-ylmethylene)bis(4,1-phenylene)diacetate, or a bioequivalent diphenylmethane. In alternative embodiments, the bisacodyl or bioequivalent diphenylmethane is formulated at or less than about 25 mg, 24 mg, 23 mg, 22 mg, 21 mg, 20 mg, 19 mg, 18 mg, 17 mg, 16 mg, 15 mg, 14 mg, 13 mg, 12 mg, 11 mg, 10 mg, 9 mg, 8 mg, 7 mg, 6 mg, 5 mg, 4 mg, 3 mg, 2 mg or 1 mg or less, or are between about 1 and 25 mg per dosage.

In alternative embodiments, a formulation or composition of the invention comprises between about 10 mg to about 1, 2, 3, 4 or 5 or more grams (g) bisacodyl, or between about 75, 80, 85, 90 or 100 mg to about 150 to 200 mg (e.g., for a normal patient) bisacodyl, or between about 100, 110, 120, 130, 140 or 150 mg to about 1, 2, 3, 4 or 4.5 g or more bisacodyl for a constipated patient.

In one embodiment, a bisacodyl or a bioequivalent diphenylmethane is used in a preparation of the invention at a final dose of about 10 mg spread over the day; this can reduce any peak dosage levels at which side effects occur and exposes the gut to much lower concentrations of bisacodyl than is currently recommended. Hence the potential for cramping or adverse effects is minimized with this formulation.

In alternative embodiments, the bisacodyl is DULCOLAX™, DUROLAX™, FLEET™, ALOPHEN™ or CORRECTOL™.

### Biofilm Disrupting Compounds

In alternative embodiments, biofilm disrupting compounds added into a composition or formulation of the invention, or used to practice a method of the invention. In alternative embodiments, disrupting biofilms are used to separate from the colonic mucosa an adherent polysaccharide/DNA-containing layer, the so-called “biofilm”, to achieve a cleaner and/or more easily visualized or stained mucosa. In alternative embodiments, bisoxatin itself is used, it has such an action in-part, achieving a cleaner caecum.

In alternative embodiments, other biofilm disrupting components or agents also can be used, e.g., enzymes such as deoxyribonuclease (DNase), N-acetylcysteine, alginate lyase, glycoside hydrolase dispersin B; Quorum-sensing inhibitors e.g., ribonucleic acid III inhibiting peptide, Salvadora persica extracts, Competence-stimulating peptide, Patulin and penicillic acid; peptides—cathelicidin-derived peptides, small lytic peptide, PTP-7 (a small lytic peptide, see e.g., Kharidia (2011) J. Microbiol. 49(4):663-8, Epub 2011 Sep. 2), Nitric oxide, neo-emulsions; ozone, lytic bacteriophages, lactoferrin, xylitol hydrogel, synthetic iron chelators, cranberry components, curcumin, silver nanoparticles, Acetyl-11-keto-β-boswellic acid (AKBA), barley coffee components, probiotics, sinefungin, S-adenosylmethionine, S-adenosyl-homocysteine, Delisea furanones, N-sulfonyl homoserine lactones and/or macrolide antibiotics or any combination thereof.

In alternative embodiments, biofilm disrupting components or agents are administered with a formulation or composition of the invention, e.g., are administered throughout or concentrated at the end of a treatment comprising a method of this invention, e.g., as a laxative.

### Unit Dosage Forms and Formulations and Delivery Vehicles

In alternative embodiments, a composition is manufactured, labeled or formulated as a liquid, a suspension, a spray, a gel, a geltab, a semisolid, a tablet, or sachet, a capsule, a lozenge, a chewable or suckable unit dosage form, or any pharmaceutically acceptable formulation or preparation. In alternative embodiments, a composition of the invention is incorporated into a food, a feed, a drink, a nutritional or a food or feed supplement (e.g., liquid, semisolid or solid), and the like.

For example, a composition of the invention can be manufactured, labeled or formulated as an orally disintegrating tablet as described e.g., in U.S. Pat. App. Publication No. 20100297031. A composition of the invention can be a polyol/thickened oil suspension as described in U.S. Pat. Nos. 6,979,674; 6,245,740. A composition of the invention can be encapsulated, e.g., encapsulated in a glassy matrix as described e.g., in U.S. Pat. App. Publication No. 20100289164; and U.S. Pat. No. 7,799,341. A composition of the invention can be manufactured, labeled or formulated as an excipient particle, e.g., comprising a cellulosic material such as microcrystalline cellulose in intimate association with silicon dioxide, a disintegrant and a polyol, sugar or a polyol/sugar blend as described e.g., in U.S. Pat. App. Publication No. 20100285164. A composition of the invention can be manufactured, labeled or formulated as an orally disintegrating tablet as described e.g., in U.S. Pat. App. Publication No. 20100278930. A composition of the invention can be manufactured, labeled or formulated as a spherical particle, as described e.g., in U.S. Pat. App. Publication No. 20100247665, e.g., comprising a crystalline cellulose and/or powdered cellulose. A composition of the invention can be manufactured, labeled or formulated as a rapidly disintegrating solid preparation useful e.g. as an orally-disintegrating solid preparation, as described e.g., in U.S. Pat. App. Publication No. 20100233278. A composition of the invention can be manufactured, labeled or formulated as a solid preparation for oral application comprising a gum tragacanth and a polyphosphoric acid or salt thereof, as described e.g., in U.S. Pat. App. Publication No. 20100226866.

A composition of the invention can be manufactured, labeled or formulated using a water soluble polyhydroxy compound, hydroxy carboxylic acid and/or polyhydroxy carboxylic acid, as described e.g., in U.S. Pat. App. Publication No. 20100222311. A composition of the invention can be manufactured, labeled or formulated as a lozenge, or a chewable and suckable tablet or other unit dosage form, as described e.g., in U.S. Pat. App. Publication No. 20100184785.

A composition of the invention can be manufactured, labeled or formulated in the form of an agglomerate, as described e.g., in U.S. Pat. App. Publication No. 20100178349. A composition of the invention can be manufactured, labeled or formulated in the form of a gel or paste, as described e.g., in U.S. Pat. App. Publication No. 20060275223. A composition of the invention can be manufactured, labeled or formulated in the form of a soft capsule, as described e.g., in U.S. Pat. No. 7,846,475, or U.S. Pat. No. 7,763,276.

The polyols used in compositions of the invention can be micronized polyols, e.g., micronized polyols, e.g., as described e.g., in U.S. Pat. App. Publication No. 20100255307, e.g., having a particle size distribution (d₅₀) of from 20 to 60 μm, and a flowability below or equal to 5 s/100 g, or below 5 s/100 g.

### Gradual or Delayed Release Formulations

In alternative embodiments, the invention provides compositions formulated for delayed or gradual enteric release comprising at least one active agent formulated with a delayed release composition or formulation, coating or encapsulation. The at least one active agent can be a bisoxatin, or a bisoxatin acetate, or an equivalent.

In alternative embodiments, compositions of the invention are formulated for delayed or gradual enteric release using cellulose acetate (CA) and polyethylene glycol (PEG), e.g., as described by Defang et al. (2005) Drug Develop. & Indust. Pharm. 31:677-685, who used CA and PEG with sodium carbonate in a wet granulation production process.

In alternative embodiments, compositions of the invention are formulated for delayed or gradual enteric release using a hydroxypropylmethylcellulose (HPMC), a microcrystalline cellulose (MCC) and magnesium stearate, as described e.g., in Huang et al. (2004) European J. of Pharm. & Biopharm. 58: 607-614).

In alternative embodiments, compositions of the invention are formulated for delayed or gradual enteric release using e.g., a poly(meth)acrylate, e.g. a methacrylic acid copolymer B, a methyl methacrylate and/or a methacrylic acid ester, a polyvinylpyrrolidone (PVP) or a PVP-K90 and a EUDRAGIT® RL PO™, as described e.g., in Kuksal et al. (2006) AAPS Pharm. 7(1), article 1, E1 to E9.

In alternative embodiments, compositions of the invention are formulated for delayed or gradual enteric release as described in U.S. Pat. App. Pub. 20100239667, which describes layered pharmaceutical compositions suitable for oral use where absorption takes place over a large part of the gastrointestinal tract. In alternative embodiments, the composition comprises a solid inner layer sandwiched between two outer layers. The solid inner layer can comprise the active agent and one or more disintegrants and/or exploding agents, one of more effervescent agents or a mixture. Each outer layer can comprise a substantially water soluble and/or crystalline polymer or a mixture of substantially water soluble and/or crystalline polymers, e.g., a polyglycol.

In alternative embodiments, compositions of the invention are formulated for delayed or gradual enteric release as described in U.S. Pat. App. Pub. 20120183612, which describes stable pharmaceutical formulations comprising active agents in a non-swellable diffusion matrix. The active agents can be released from the matrix in a sustained, invariant and, if several active agents are present, independent manner and the matrix is determined with respect to its substantial release characteristics by ethylcellulose and at least one fatty alcohol.

In alternative embodiments, compositions of the invention are formulated for delayed or gradual enteric release as described in U.S. Pat. No. 6,284,274, which describes a bilayer tablet containing an active agent (e.g., an opiate analgesic), a polyalkylene oxide, a polyvinylpyrrolidone and a lubricant in the first layer and a second osmotic push layer containing polyethylene oxide or carboxymethylcellulose.

In alternative embodiments, compositions of the invention are formulated for delayed or gradual enteric release as described in U.S. Pat. App. Pub. No. 20030092724, which describes sustained release dosage forms in which a nonopioid analgesic and opioid analgesic are combined in a sustained release layer and in an immediate release layer, sustained release formulations comprising microcrystalline cellulose, EUDRAGIT RSPO™, CAB-O-SIL™, sodium lauryl sulfate, povidone and magnesium stearate.

In alternative embodiments, compositions of the invention are formulated for delayed or gradual enteric release as described in U.S. Pat. App. Pub. 20080299197, describing a multi-layered tablet for a triple combination release of active agents to an environment of use, e.g., in the GI tract. In alternative embodiments, a multi-layered tablet is used, and it can comprise two external drug-containing layers in stacked arrangement with respect to and on opposite sides of an oral dosage form that provides a triple combination release of at least one active agent. In one embodiment the dosage form is an osmotic device, or a gastro-resistant coated core, or a matrix tablet, or a hard capsule.

In alternative embodiments, compositions of the invention are formulated as multiple layer tablet forms, e.g., where a first layer provides an immediate release of an active agent and a second layer provides a controlled-release of another (or the same) active agent, as described e.g., in U.S. Pat. No. 6,514,531 (disclosing a coated trilayer immediate/prolonged release tablet), U.S. Pat. No. 6,087,386 (disclosing a trilayer tablet), U.S. Pat. No. 5,213,807 (disclosing an oral trilayer tablet with a core comprising an active agent and an intermediate coating comprising a substantially impervious/impermeable material to the passage of the first active agent), and U.S. Pat. No. 6,926,907 (disclosing a trilayer tablet that separates a first active agent contained in a film coat from a core comprising a controlled-release second active agent formulated using excipients which control the drug release, the film coat can be an enteric coating configured to delay the release of the active agent until the dosage form reaches an environment where the pH is above four).

In alternative embodiments, compositions of the invention are formulated for delayed or gradual enteric release as described in U.S. Pat. App. Pub. 20120064133, which describes a release-retarding matrix material such as: an acrylic polymer, a cellulose, a wax, a fatty acid, shellac, zein, hydrogenated vegetable oil, hydrogenated castor oil, polyvinylpyrrolidine, a vinyl acetate copolymer, a vinyl alcohol copolymer, polyethylene oxide, an acrylic acid and methacrylic acid copolymer, a methyl methacrylate copolymer, an ethoxyethyl methacrylate polymer, a cyanoethyl methacrylate polymer, an aminoalkyl methacrylate copolymer, a poly(acrylic acid), a poly(methacrylic acid), a methacrylic acid alkylamide copolymer, a poly(methyl methacrylate), a poly(methacrylic acid anhydride), a methyl methacrylate polymer, a polymethacrylate, a poly(methyl methacrylate) copolymer, a polyacrylamide, an aminoalkyl methacrylate copolymer, a glycidyl methacrylate copolymer, a methyl cellulose, an ethylcellulose, a carboxymethylcellulose, a hydroxypropylmethylcellulose, a hydroxymethyl cellulose, a hydroxyethyl cellulose, a hydroxypropyl cellulose, a crosslinked sodium carboxymethylcellulose, a crosslinked hydroxypropylcellulose, a natural wax, a synthetic wax, a fatty alcohol, a fatty acid, a fatty acid ester, a fatty acid glyceride, a hydrogenated fat, a hydrocarbon wax, stearic acid, stearyl alcohol, beeswax, glycowax, castor wax, carnauba wax, a polylactic acid, polyglycolic acid, a co-polymer of lactic and glycolic acid, carboxymethyl starch, potassium methacrylate/divinylbenzene copolymer, crosslinked polyvinylpyrrolidone, polyvinylalcohols, polyvinylalcohol copolymers, polyethylene glycols, non-crosslinked polyvinylpyrrolidone, polyvinylacetates, polyvinylacetate copolymers or any combination. In alternative embodiments, spherical pellets are prepared using an extrusion/spheronization technique, of which many are well known in the pharmaceutical art.

In alternative embodiments, compositions of the invention are formulated for delayed or gradual enteric release as described in U.S. Pat. App. Pub. 20110218216, which describes an extended release pharmaceutical composition for oral administration, and uses a hydrophilic polymer, a hydrophobic material and a hydrophobic polymer or a mixture thereof, with a microenvironment pH modifier. The hydrophobic polymer can be ethylcellulose, cellulose acetate, cellulose propionate, cellulose butyrate, methacrylic acid-acrylic acid copolymers or a mixture thereof. The hydrophilic polymer can be polyvinylpyrrolidone, hydroxypropylcellulose, methylcellulose, hydroxypropylmethyl cellulose, polyethylene oxide, acrylic acid copolymers or a mixture thereof. The hydrophobic material can be a hydrogenated vegetable oil, hydrogenated castor oil, carnauba wax, candellia wax, beeswax, paraffin wax, stearic acid, glyceryl behenate, cetyl alcohol, cetostearyl alcohol or and a mixture thereof. The microenvironment pH modifier can be an inorganic acid, an amino acid, an organic acid or a mixture thereof. Alternatively, the microenvironment pH modifier can be lauric acid, myristic acid, acetic acid, benzoic acid, palmitic acid, stearic acid, oxalic acid, malonic acid, succinic acid, adipic acid, sebacic acid, fumaric acid, maleic acid; glycolic acid, lactic acid, malic acid, tartaric acid, citric acid, sodium dihydrogen citrate, gluconic acid, a salicylic acid, tosylic acid, mesylic acid or malic acid or a mixture thereof.

### Feeds, Drinks, Candies, Nutritional or a Food or Feed Supplements

In alternative embodiments, a composition of the invention is incorporated into a food, a feed, a candy (e.g., a lollypop or a lozenge) a drink, a nutritional or a food or feed supplement (e.g., liquid, semisolid or solid), and the like, as described e.g., in U.S. Pat. App. Publication No. 20100178413. In one embodiment, a composition of the invention is incorporated into (manufactured as) a beverage as described e.g., in U.S. Pat. No. 7,815,956. For example, a composition of the invention is incorporated into a yogurt, an ice cream, a milk or milkshake, a “frosty”, “snow-cone”, or other ice-based mix, and the like.

### Osmotic Laxatives, Polyethylene Glycols (PEGs)

In alternative embodiments, compositions and methods of the invention comprise use of a bisoxatin (or 2,2-bis(4-hydroxyphenyl)-2H-benzo[b][1,4]oxazin-3(4H)-one), or a bisoxatin acetate, or an equivalent, and an osmotic laxative. In alternative embodiments, the osmotic laxative comprises a sorbitol, mannitol, lactulose and/or a polyethylene glycol, or an equivalent non-absorbable, non-metabolized osmotic agent; wherein optionally the polyethylene glycol (PEG) is a PEG 3350, or MIRALAX™. In alternative embodiments, this combination can further comprise a third or an additional agent, such as an antibiotic or an antimicrobial, or a vitamin, such as vitamin C. In alternative embodiments, these combinations of the invention are used to treat, ameliorate, reverse or prevent a constipation, e.g., an occasional constipation, a chronic constipation, or a severe constipation, or a constipation secondary to a drug use (e.g., a narcotic) or a condition as described herein.

In one embodiment, a PEG dosage in the combination is about 17 grams, which is about one heaping tablespoon of powder of PEG 3350, or MIRALAX™. The PEG 3350, or MIRALAX™ can be dissolved in four to eight ounces of liquid and swallowed once a day. In one embodiment, the combination of the invention is formulated as a single-dose packet, or sachet, containing about 17 grams, of PEG 3350, or MIRALAX™ powder. Patients can be instructed to use a single-dose packet, dissolving an entire packet in four to eight ounces of a liquid.

In alternative embodiments, patients can be instructed to initially take two sachets containing either PEG (13 g/sachet) or lactulose (10 g/sachet), and can be given an option to change the dose to one or three sachets/day, depending on response.

In alternative embodiments, a mean initial dose of PEG in the combination is about 0.88 g/kg/day, or ranging from about 0.26-2.14 g/kg/day; and a mean effective maintenance dose of PEG in the combination can be about 0.78 g/kg/day, or in a range from about 0.26-1.30 g/kg/day.

In alternative embodiments, this embodiment combination is administered by mouth, e.g., usually once daily, or as needed depending on the condition of the patient or the condition treated. In alternative embodiments, individual packets, e.g., sachets with powder are used. In alternative embodiments, a container or a bottle is used, and a cap can be included to measure the appropriate or prescribed dose.

In alternative embodiments, this embodiment combination can be mixed (e.g., the powder can be mixed) with a full glass (e.g., about 8 ounces or 240 milliliters) of a liquid such as a water, juice, soda, coffee, or tea. In alternative embodiments, this embodiment combination is taken for about 2 to 4 days to about two weeks or until a bowel movement. Do not increase your dose or take it more frequently than prescribed.

### Packaging

The invention provides compositions, including preparations, formulations and/or kits, comprising combinations of ingredients, as described herein (e.g., a bisoxatin and an anti-inflammatory agent, a bisoxatin and an osmotic laxative, a bisoxatin, a laxative and an antibiotic, a bisoxatin and an olsalazine). In one aspect, each member of the combination of ingredients is manufactured in a separate package, kit or container; or, all or a subset of the combinations of ingredients are manufactured in a separate package or container. In alternative aspects, the package, kit or container comprises a blister package, a clamshell, a tray, a shrink wrap and, the like.

In one aspect, the package, kit or container comprises a “blister package” (also called a blister pack, or bubble pack). In one aspect, the blister package is made up of two separate elements: a transparent plastic cavity shaped to the product and its blister board backing. These two elements are then joined together with a heat sealing process which allows the product to be hung or displayed. Exemplary types of “blister packages” include: Face seal blister packages, gang run blister packages, mock blister packages, interactive blister packages, slide blister packages.

Blister packs, clamshells or trays are forms of packaging used for goods; thus, the invention provides for blister packs, clamshells or trays comprising a composition (e.g., a (the multi-ingredient combination of drugs of the invention) combination of active ingredients) of the invention. Blister packs, clamshells or trays can be designed to be non-reclosable, so consumers can tell if a package has already opened. They are used to package for sale goods where product tampering is a consideration, such as the pharmaceuticals of the invention. In one aspect, a blister pack of the invention comprises a moulded PVC base, with raised areas (the “blisters”) to contain the tablets, pills, etc. comprising the combinations of the invention, covered by a foil laminate. Tablets, pills, etc. are removed from the pack either by peeling the foil back or by pushing the blister to force the tablet to break the foil. In one aspect, a specialized form of a blister pack is a strip pack. In one aspect, in the United Kingdom, blister packs adhere to British Standard 8404.

In one embodiment, the invention also provides a method of packaging where the compositions comprising combinations of ingredients of the invention are contained in-between a card and a clear PVC. The PVC can be transparent so the item (pill, tablet, geltab, etc.) can be seen and examined easily; and in one aspect, can be vacuum-formed around a mould so it can contain the item snugly and have room to be opened upon purchase. In one aspect, the card is brightly colored and designed depending on the item (pill, tablet, geltab, etc.) inside, and the PVC is affixed to the card using pre-formed tabs where the adhesive is placed. The adhesive can be strong enough so that the pack may hang on a peg, but weak enough so that this way one can tear open the join and access the item. Sometimes with large items or multiple enclosed pills, tablets, geltabs, etc., the card has a perforated window for access. In one aspect, more secure blister packs, e.g., for items such as pills, tablets, geltabs, etc. of the invention are used, and they can comprise of two vacuum-formed PVC sheets meshed together at the edges, with the informative card inside. These can be hard to open by hand, so a pair of scissors or a sharp knife may be required to open.

In one aspect, blister packaging comprises at least two or three or more components (e.g., is a multi-ingredient combination of the invention): a thermoformed “blister” which houses multi-ingredient combination of the invention, and then a “blister card” that is a printed card with an adhesive coating on the front surface. During the assembly process, the blister component, which is most commonly made out of PVC, is attached to the blister card using a blister machine. This machine introduces heat to the flange area of the blister which activates the glue on the card in that specific area and ultimately secures the PVG blister to the printed blister card. The thermoformed PVG blister and the printed blister card can be as small or as large as you would like, but there are limitations and cost considerations in going to an oversized blister card. Conventional blister packs can also be sealed (e.g., using an AERGO 8 DUO™, SCA Consumer Packaging, Inc., DeKalb Ill.) using regular heat seal tooling. This alternative aspect, using heat seal tooling, can seal common types of thermoformed packaging.

### Blister Packaging

In alternative embodiments, combinations of the invention can comprise the packaging of the therapeutic drug combinations of the invention, alone or in combination, as “blister packages” or as a plurality of packettes, including as lidded blister packages, lidded blister or blister card or packets or packettes, or a shrink wrap.

In alternative embodiments, laminated aluminum foil blister packs are used, e.g., for the preparation of drugs designed to dissolve immediately in the mouth of a patient. This exemplary process comprises having the drug combinations of the invention prepared as an aqueous solution(s) which are dispensed (e.g., by measured dose) into an aluminum (e.g., alufoil) laminated tray portion of a blister pack. This tray is then freeze-dried to form tablets which take the shape of the blister pockets. The alufoil laminate of both the tray and lid fully protects any highly hygroscopic and/or sensitive individual doses. In one aspect, the pack incorporates a child-proof peel open security laminate. In one aspect, the system give tablets an identification mark by embossing a design into the alufoil pocket that is taken up by the tablets when they change from aqueous to solid state. In one aspect, individual ‘push-through’ blister packs/packettes are used, e.g., using hard temper aluminum (e.g., alufoil) lidding material. In one aspect, hermetically-sealed high barrier aluminum (e.g., alufoil) laminates are used. In one aspect, any of the invention's products of manufacture, including kits or blister packs, use foil laminations and strip packs, stick packs, sachets and pouches, peelable and non-peelable laminations combining foil, paper, and film for high barrier packaging.

In alternative embodiments, any of the invention's multi-ingredient combinations or products of manufacture, including kits or blister packs, include memory aids to help remind patients when and how to take the drug. This safeguards the drug's efficacy by protecting each pill until it's taken; gives the product or kit portability, makes it easy to take a dose anytime or anywhere.

The invention will be further described with reference to the following examples; however, it is to be understood that the invention is not limited to such examples.

### EXAMPLES

### Example 1

### Bisoxatin Micro-Encapsulated Granules Preparations of the Invention for Constipation

This example demonstrates that bisoxatin preparations of the invention are effective in patients.

A 42 year old female with lifelong constipation, not defecating up to 2 weeks, was assessed endoscopically with stool tests and other investigations. Having failed various laxatives over the years and, she had not previously used bisoxatin as a graded release.

A bisoxatin preparation with micro-encapsulated granules were separately encapsulated and administered. These capsules are formulated to open either only in the upper distal small bowel or the colon. The formulation per capsule contains 60 mg of bisoxatin and she initially started with 60 mg per day but required 60 mg twice daily. After about 5 days she started having looser motions and defecated up to 3 or 4 times per day upon which she reduced the dose to only 60 mg per day. However when she went on an overseas trip she had to use three 60 mg capsules per day to actually defecate effectively. After 3 months of usage she was able to alter the medications adequately to have one to three stools per day.

### Example 2

### Bisoxatin Preparations of the Invention for Parkinson's Disease

This example demonstrates that exemplary bisoxatin preparations of the invention, e.g., a slow release Bisoxatin capsule, are effective in patients, including Parkinson's Disease patients.

A patient with Parkinson's Disease and long standing constipation was referred for treatment of his dysmotility. He was given a slow release Bisoxatin capsule which released generally in the distal small bowel. The capsule was enteric coated and contained 120 mg of bisoxatin. The patient did not defecate spontaneously prior to usage of the bisoxatin. He had only enema therapies otherwise he would not defecate at all. The 120 mg slow release capsule gave him second daily defecation and for several weeks upon which time he decided to use 2 capsules per day and then alternated between one and two capsules per day which gave him good steady defecation.

### Example 3

### Bisoxatin Preparations of the Invention for Constipation

This example demonstrates that bisoxatin preparations of the invention, e.g., comprising bisoxatin and rifaximin, are effective in patients with severe constipation.

A 62 year old female patient with “constipation as long as I can remember” presented for treatment. She would not defecate for up to a week and suffered with associated bloating. Gurgling and wind were other symptoms. She was commenced on capsulated combination of bisoxatin 30 mg and rifaximin 500 mg to be taken twice daily. The combination started her defecating within 2 days very efficiently and she had to slow down the medication to one capsule per day and continued on for 3 months with excellent defecation qualities. Her bloat progressively came down and she was no longer complaining of gurgling and excessive wind. She made a particular comment that this was the first time that her bowels worked so well when compared with other laxatives which she had used throughout her life.

### Example 4

### Bisoxatin Preparations of the Invention for Constipation

This example demonstrates that bisoxatin preparations of the invention, e.g., bisoxatin and vancomycin, are effective in patients with severe constipation.

A 45 year old police officer presented with severe constipation not responding to many laxatives he had previously been prescribed. His main stay was 6 to 7 sachets of MOVICOL™, a PEG-based laxative. Even with that he was sensing incomplete evacuation. He was prescribed a combination of bisoxatin 25 mg and vancomycin 250 mg twice daily. On taking the medication it took 3 days until his bowel function became more frequent and initially he would defecate 4 to 5 times per day. Within a week the defecation pattern reduced down to 2 defecations and in particular he noticed that bloating and wind that he was passing had subsided considerably. He was able to continue on, the treatment and at 2 months review had a very adequate defecation pattern ranging from one and three per day.

### Example 5

### Bisoxatin Preparations of the Invention for Ulcerative Colitis

This example demonstrates that bisoxatin preparations of the invention, e.g., bisoxatin and balsalaside, are effective in Ulcerative Colitis patients.

A 28 year old patient with 6 year history of Ulcerative Colitis in combination with constipation presented for treatment. She had previously been taking azulfidine and later mesalazine for her colitis. Although the Colitis improved quite markedly with various other anti-colitis medications, as the colitis improved the patients constipation set in. Added to her other medications not listed here she was commenced on a combination of balsalaside (COLAZIDE™) 750 mg combined with bisoxatin 30 mg. These capsules were taken twice daily. This combination of the anti-inflammatory agent and the anti-constipation agent allowed her adequate defecation while also delivering an anti-inflammatory agent. She was able to continue with her other medications and without the added symptom of constipation. The intermittent bleeding that she previously continued to have possibly due to straining now settled.

### Example 6

### Bisoxatin Preparations of the Invention for Chronic Constipation

This example demonstrates that bisoxatin preparations of the invention, e.g., bisoxatin and domperidone, are effective in patients with chronic constipation.

A 68 year old female patient with chronic constipation for many years together with abdominal pain, nausea and bloating presented for treatment. She had in the past trialed a number of medications but the nausea was the overwhelming symptom. In spite of the fact that she could take various combinations of laxatives and defecate the nausea was hard to control. She was commenced on a capsule combining 30 mg of bisoxatin together with 10 mg domperidone to be taken twice daily. The patient's defecation improved quite dramatically but in addition while she was taken two capsules per day and defecating 3 times per day her nausea was decreased by “80%” by the patient's assessment. She was later given a combination of bisoxatin 30 mg and rifaximin 500 mg in a single capsule again twice daily and was able to continue with even further reduction in her nausea. She remained on a treatment for over 6 months.

Further Examples include:

### Example 7

A 42 year old patient with chronic constipation who did not respond to colchicine alone taking 1 mg in the morning and 1.5 in the evening with increased defecation was still missing a day here and there. She was given added bisoxatin of 120 mg twice daily. After two days the patient was defecating on a daily basis. She was going too well however and the bisoxatin had to be cut back 120 mg mane. For a week or so she was able to take the bisoxatin 120 in the morning and every second day 120 at night. She continued with the treatment for 3 or 4 weeks to date.

### Example 8

A 21 year old hairdresser with long history of mild constipation not responding to metamucil and benefiber was seen for further treatment. After investigations with colonoscopy and cultures she was prescribed 60 mg of bisoxatin twice daily and reviewed at 2 weeks. 60 bd of bisoxatin did not give her an adequate enough response and it was raised to 120 in the morning and 60 at night. When first starting on the 120 mg she developed watery stools but after 2 or 3 days the 120 morning and 60 at night resulted in satisfactory emptying of the bowel with the added fibre product. She continued for 4 weeks on the treatment before the medications were adjusted to cope with the fluctuating constipation. She would then take 120 in the morning and no capsule at night of bisoxatin or 120 in the morning and 60 at night adjusting per dosage on the response to defecation.

### Example 9

This patient with mild constipation missing a stool every second or third day was treated with the bisoxatin alone. He was given bisoxatin 60 bd and felt within 24 hours an improvement in defecation which was incomplete but nevertheless much better than before. To improve his defecation capacity the bisoxatin 60 bd was combined with naloxone hydrochloride 30 mg bd in an enteric coated capsule. From about day 3 of starting the new medication he noticed a more complete emptying which persisted at 4 weeks where the medication trial was stopped.

### Example 10

Bisoxatin and a ‘Biofilm disrupting agent’—olsalazine. In this patient bisoxatin was commenced to treat moderate constipation. She had the disorder for about 15 years and had been normally using Chinese and Indian teas to defecate every 2 or 3 days. She was given bisoxatin 60 bd which did not work initially and had to continue the teas. The bisoxatin was raised to 120 bd and at 5 days she started defecating better but still incompletely. She was then given a Biofilm disrupting agent. Olsalazine 500 mg bd and increased to 1 gm bd, and by the end of the week she was defecating much more satisfactorily with large full stools (Bristol Chart 3).

A number of embodiments of the invention have been described. Nevertheless, it will be understood that various modifications may be made without departing from the spirit and scope of the invention. Accordingly, other embodiments are within the scope of the following claims.

1-54. (canceled)

55. A composition formulated for delayed release, comprising a bisoxatin (or 2,2-bis(4-hydroxyphenyl)-2H-benzo[b][1,4]oxazin-3(4H)-one), or a bisoxatin acetate, and an enteric coating.

56. The composition of claim 55, wherein the enteric coating comprises a gastro-resistant coating that dissolves at a pH of 7 in the terminal ileum.

57. The composition of claim 55, wherein the enteric coating comprises a poly(meth)acrylate, a methyl methacrylate and/or a methacrylic acid ester.

58. The composition of claim 55, wherein the enteric coating comprises: a vinyl or a polyvinyl acetate phthalate; a hydroxypropylmethylcellulose (HPMC); a high viscosity grade HPMC; an ultra-high viscosity grade HPMC; a polyvinylpyrrolidone (PVP); a PVP-90; a cellulose; a microcrystalline cellulose (MCC); a methylcellulose; a hydroxy methylcellulose; an ethyl cellulose; and a copolymer of ethyl acrylate, methyl methacrylate and a methacrylic acid ester with quaternary ammonium groups, or any combination or mixture thereof.

59. The composition of claim 55, wherein the enteric coating comprises: cellulose acetate phthalate, hydroxypropyl methylcellulose phthalate, polyvinyl acetate phthalate, hydroxypropyl methylcellulose acetate succinate, cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methyl vinyl ether and maleic anhydride, ethyl methyacrylate-methylmethacrylate-chlorotrimethylammonium ethyl acrylate copolymer, natural resins, zein, shellac, copal collophorium or an acrylic copolymer, or any combination or mixture thereof.

60. The composition of claim 55, wherein the enteric coating comprises (i) a wax and (ii) at least one of: a glyceryl monostearate, a stearic acid, a palmitic acid, a glyceryl monopalmitate, a cetyl alcohol, a shellac, a zein, an ethylcellulose, an acrylic resin, a cellulose acetate or a silicone elastomer or any combination or mixture thereof.

61. The composition of claim 55, comprising between about 100 mg to about 4.5 g of bisoxatin or bisoxatin acetate.

62. The composition of claim 55, comprising between about 0.10 to about 1000 milligrams (mg) of bisoxatin or bisoxatin acetate.

63. The composition of claim 55, comprising between about 10 mg to about 500 mg bisoxatin or bisoxatin acetate.

64. The composition of claim 55, comprising between about 50 mg to about 200 mg of bisoxatin or bisoxatin acetate.

65. The composition of claim 55, comprising between about 100 to 250 mg of bisoxatin or bisoxatin acetate.

66. The composition of claim 55, comprising about 60 mg bisoxatin or bisoxatin acetate.

67. The composition of claim 55, comprising about 120 mg of bisoxatin or bisoxatin acetate.

68. The composition of claim 55, wherein the composition comprises an enteric coated tablet, a multi-particulate or multilayered tablet or capsule; or a gelatin, a soft gelatin or equivalent thereof.

69. The composition, pharmaceutical composition or formulation of claim 55, further comprising a water-soluble salt selected from the group consisting of: a calcium salt, a calcium carbonate, a calcium acetate, a citrate salt, a calcium citrate, a magnesium salt, a magnesium sulphate, a magnesium citrate, a monobasic sodium phosphate, dibasic sodium phosphate, and/or tribasic sodium phosphate, a magnesium phosphate, a sodium salt, a sodium sulphate, a sodium chloride, a sodium gluconate, a sodium citrate, a sodium aspartate, a potassium salt, a potassium gluconate, a potassium tartrate, a potassium chloride, an acetate salt, an adipate salt, an alginate salt, an aspartate salt, a benzoate salt, a benzenesulfonate salt, a bisulfate salt, a butyrate salt, a camphorate salt, a camphor sulfonate salt, a digluconate salt, a glycerophosphate salt, a hemisulfate salt, a heptanoate salt, a hexanoate salt, a fumarate salt, a hydrochloride salt, a hydrobromide salt, a hydroiodide salt, a 2-hydroxyethansulfonate (isothionate) salt, a lactate salt, a maleate salt, a methane sulfonate salt, a nicotinate salt, a 2-naphthalene sulfonate salt, an oxalate salt, a palmitoate salt, a pectinate salt, a persulfate salt, a 3-phenylpropionate salt, a picrate salt, a pivalate salt, a propionate salt, a succinate salt, a tartrate salt, a thiocyanate salt, a phosphate salt, a glutamate salt, a bicarbonate salt, a p-toluenesulfonate salt, a undecanoate salt, or any equivalent salt, or any mixture thereof.

70. The composition, pharmaceutical composition or formulation of claim 55, further comprising at least one of:
(i) an antibiotic or an antimicrobial;
(ii) a colchicine or an equivalent thereof;
(iii) an anti-inflammatory agent;
(iv) a fiber product;
(v) a prokinetic agent;
(vi) a sulphate;
(vii) a phosphate;
(viii) a laxative;
(ix) an osmotic laxative;
(x) a non-osmotic purgative;
(xi) an anti-narcotic agent and/or a neural stimulant;
(xii) an opiate inhibitor or opiate antagonist;
(xiii) an acid suppressant, antacid and/or proton pump inhibitor;
(xiv) a probiotic; and
(xv) a Biofilm Disrupting Compound.

71. A method for the amelioration, treatment and/or prevention of:
a constipation
comprising: administering the composition of claim 55 to an individual in need thereof.

72. The method of claim 71, wherein the constipation is one or more of: functional constipation, Irritable Bowel Syndrome (IBS)-constipation, diverticulosis-associated constipation, pseudo obstruction, slow-transit constipation, stasis with overflow and diabetic gastro-paresis.

73. The method of claim 71, wherein a symptom or condition associated with constipation includes one or more of: cyclic vomiting, reflux oesophagitis, autism enteropathy, flatulence, halitosis, Chronic Fatigue Syndrome (CFS), bloating, proctalgia fugax, small intestinal bacterial overgrowth (SIBO) and large intestinal bacterial overgrowth (LIBO), chronic nausea, functional dyspepsia, and bloating.

74. The method of claim 71, wherein the bisoxatin (or 2,2-bis(4-hydroxyphenyl)-2H-benzo[b][1,4]oxazin-3(4H)-one) or bisoxatin acetate is administered at a dosage of between about 1 to 360 mg a day.

75. The method of claim 71, wherein the unit dosage of the bisoxatin (or 2,2-bis(4-hydroxyphenyl)-2H-benzo[b][1,4]oxazin-3(4H)-one) or bisoxatin acetate is between about 20 and 125 mg per unit dosage.

76. A package or kit comprising combination of at least two formulations, wherein a first formulation is contained in a first container and a second formulation is contained in a second container, and the formulations are designed to be taken in sequence as part of a treatment or a regimen, wherein a patient is administered or instructed to take the contents of the first container comprising a composition of claim 55 before the contents of the second container.
